(19) United States
(12) Patent Application Publication
(10) Pub. No.: US 2010/0280238 A1
(43) Pub. Date: Nov. 4, 2010

(54) INTRAMOLECULAR AZIDE-ALKYNE CYCLOADDITION

(76) Inventors: Lisa A. Marcaurelle, Arlington, MA (US); Ann R. Kelly, Somerville, MA (US); Sarathy Kesavan, Quincy, MA (US); Jingqiang Wei, Cambridge, MA (US); Damian W. Young, Boston, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/769,891

(22) Filed: Apr. 29, 2010

Related U.S. Application Data
(60) Provisional application No. 61/173,827, filed on Apr. 29, 2009.

(51) Int. Cl. C07D 498/18; C07D 498/14; C07D 498/04; C07D 498/08

(52) U.S. Cl. 540/456; 540/455

(57) ABSTRACT

The Huisgen 1,3-dipolar cycloaddition is a ‘click’ reaction that results from the ligation of azides and alkynes to give a triazole moiety. This reaction has been shown to be effective in the formation of a variety of macrocyclic rings. A key point of interest is the regioselectivity and specificity of the cycloaddition. Disclosed herein are specific, selective, and high-yielding methods of azide-alkyne macrocyclization to form 1,4- and 1,5-triazoles and libraries thereof.

### RELATED APPLICATIONS

This application claims the benefit of priority to U.S. Provisional Patent Application Ser. No. 61/173,827, filed Apr. 29, 2009; the contents of which are hereby incorporated by reference.

### GOVERNMENT SUPPORT

This invention was made with government support under Grant No. 5-P50-GM069721-06, awarded by the National Institutes of Health. The government has certain rights in this invention.

### BACKGROUND OF THE INVENTION

The preparation and screening of small molecules constitutes a powerful strategy for the discovery of biological probes and pharmaceutical agents. Diversity of structure within a particular compound collection is key to the discovery of hits over a wide range of biological areas. It has recently been shown that large screening collections that lack diversity are insufficient to provide lead compounds against a range of antibacterial targets. A current strategy for achieving diverse compound collections through diversity-oriented synthesis (DOS) focuses on the use of functional group pairing. By using scaffolds with multiple functional group “handles” and joining them in a pairwise, intramolecular, and chemoselective fashion both skeletal diversity and rigidity are achieved. A complementary approach for generating structural diversity is known as “reagent-based” diversification. This strategy involves the preparation of a singular scaffold that, when subjected to different reaction conditions, selectively yields different products. To further develop this strategy, robust methodologies that allow for reagent-based differentiation must be developed.

The Huisgen 1,3-dipolar cycloaddition is a ‘click’ reaction that results from the ligation of azides and alkynes to give a triazole moiety. This reaction has been shown to be effective in the formation of a variety of macrocyclic rings. A key point of interest is the regioselectivity of the cycloaddition. While advances have been made in the formation of 1,4-triazoles using copper (I) catalysis, the formation of 1,5-triazole rings using ruthenium (II) catalysis remains challenging.

Recently, a macrocyclization using azide-alkyne reactions was attempted; unfortunately, copper-catalyzed macrocyclization of an azide-alkyne tetrapeptide produced the desired 1,4-triazole product in only 50% yield. Horne, W. S. et al. “Probing the Bioactive Conformation of an Archetypal Natural Product HDAC Inhibitor with Conformationally Homogeneous Triazole-Modified Cyclic Tetrapeptides.” Angew. Chem. Int. Ed. 2009, 48. Furthermore, in an attempt to synthesize the 1,5-triazole product via a thermal cyclization reaction, a 2:1 mixture of 1,5- and 1,4-triazoles was obtained. In this case, the desired 1,5-triazole was obtained in only 8% isolated yield. The authors described the failure of a Ru-catalyzed reaction in forming the desired 1,5-isomer, and resorted to an alternative method of macrocyclization; the 1,5-triazole moiety was formed first, then the linear molecule was cyclized in a macrolactamization reaction.

Consequently, a need exists for a specific, selective, and high-yielding method of azide-alkyne macrocyclization to form 1,4- and 1,5-triazoles and libraries thereof.

### SUMMARY OF THE INVENTION

In certain embodiments, the invention relates to a method of forming a triazole according to Scheme 1:

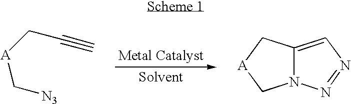

wherein, independently for each occurrence,

A is -(a)ₘ-;

metal catalyst consists essentially of at least one ligand and Ru;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to a method of forming a triazole according to Scheme 2:

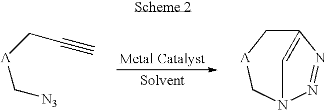

wherein, independently for each occurrence,

A is -(a)ₘ-;

metal catalyst consists essentially of at least one ligand and Cu;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to a method of forming a triazole according to Scheme 3:

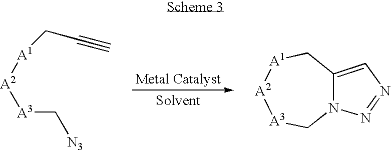

wherein, independently for each occurrence,

A¹ is —(CR₂)ₙ—, —(CR₂)ₙ—O—, —O—(CR₂)ₙ—, —O—(CR₂)ₙ—O—, —(CR₂)ₙ—NR—;

A² is —NR—C(═O)—, —C(═O)—NR—, or

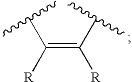

A³ is —(CR₂)ₙ—;

metal catalyst consists essentially of at least one ligand and Ru;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to a method of forming a triazole according to Scheme 4:

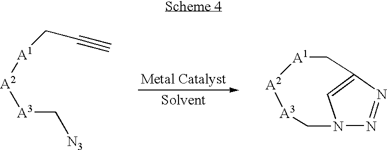

wherein, independently for each occurrence,

A¹ is —(CR₂)ₙ—, —(CR₂)ₙ—O—, —O—(CR₂)ₙ—, —O—(CR₂)ₙ—O—, —(CR₂)ₙ—NR—;

A² is —NR—C(O)—, —C(O)—NR—, or

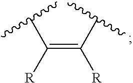

A³ is —(CR₂)ₙ—;

metal catalyst consists essentially of at least one ligand and Cu;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein one any of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to a compound of formula I or formula II

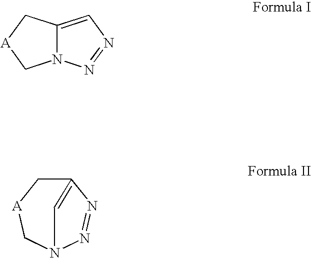

or a pharmaceutically acceptable salt thereof,
wherein, independently for each occurrence,

A is -(a)ₘ-;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to a compound of formula III or formula IV

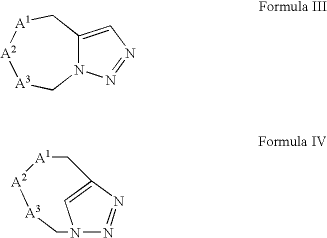

or a pharmaceutically acceptable salt thereof,
wherein, independently for each occurrence,

A¹ is —(CR₂)ₙ—, —(CR₂)ₙ—O—, —O—(CR₂)ₙ—, —O—(CR₂)ₙ—O—, —(CR₂)ₙ—NR—;

A² is —NR—C(═O)—, —C(═O)—NR—, or

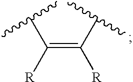

A³ is —(CR₂)ₙ—;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to a library comprising a plurality of compounds of formula I and a plurality of compounds of formula II.

In certain embodiments, the invention relates to a library comprising a plurality of compounds of formula III and a plurality of compounds of formula IV.

### BRIEF DESCRIPTION OF THE FIGURES

FIG. 1 depicts the formation of regioisomeric triazoles in an intramolecular Huisgen cycloaddition.

FIG. 2 depicts an exemplary synthesis of an alkyne-azide and an intramolecular Ru-catalyzed cycloaddition thereof.

FIG. 3 depicts a table outlining an optimization of the reaction conditions in a Ru-catalyzed cycloaddition reaction.

FIG. 4 depicts the X-ray crystal structures of representative macrocyclic triazoles (4a and 5f).

FIG. 5 depicts an exemplary cycloaddition reaction scheme and a table outlining an optimization of the reaction conditions using various copper catalysts.

FIG. 6 depicts a table outlining the results for exemplary intramolecular cycloadditions with linear and cyclic substrates.

FIG. 7 depicts a table summarizing data illuminating the influence of stereochemistry on the outcome of exemplary intramolecular cycloadditions.

FIG. 8 depicts a table outlining the results for exemplary intramolecular cycloadditions with linear and cyclic substrates.

FIG. 9 depicts the synthesis of an azido-alkyne substrate (3l).

FIG. 10 depicts exemplary regioselective alkyne-azide macrocyclization reactions.

FIG. 11 depicts the effect of the use of polymer-bound Cu catalysts on the outcome of an exemplary alkyne-azide cycloaddition.

FIG. 12 depicts the ratios of monomer (intramolecular reaction product) to dimer (intermolecular reaction product) produced in a solution-phase reaction and two reactions using polymer-bound catalyst. Use of a polymer-bound catalyst decreases the amount of dimerization (cf. FIG. 11).

FIG. 13 depicts a flow reactor that may be used with a solid-supported catalyst.

FIG. 14 depicts an exemplary solid supported copper catalyst. When loaded, the resin turns green.

FIG. 15 depicts the results showing the influence of stereocenters in the tether between the alkyne and azide on the outcome of a macrocyclization reaction of the present invention.

FIG. 16 depicts exemplary post-macrocyclization transformations.

FIG. 17 depicts an exemplary synthesis of a library of diverse molecules from a product of an intramolecular alkyne-azide cycloaddition.

### DETAILED DESCRIPTION OF THE INVENTION

### Overview

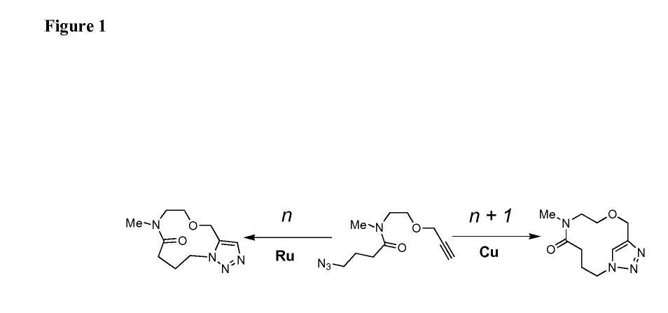

In certain embodiments, the invention relates to a method of regioselectively synthesizing macrocyclic triazole rings. The method is suited to the preparation of small-molecule libraries because one compound can be converted into two structurally unique macrocycles that have an n or n+1 ring size (FIG. 1). Access to structurally related pairs of macrocyclic triazoles could provide insight into their antibacterial and cytotoxic biological activity, two areas in which triazole-containing small molecules have shown promise. Furthermore, the methods of the present invention help to develop an understanding of which substrates and ring sizes provide the best yields. Additionally, the method may be used in the synthesis of combinatorial libraries of regioisomeric triazoles.

### DEFINITIONS

For convenience, before further description of the disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “acyl” as used herein refers to the radical

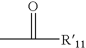

wherein R′₁₁ represents hydrogen, alkyl, alkenyl, alkynyl, or —(CH₂)ₘ—R₈₀, wherein R₈₀ is aryl, cycloalkyl, cycloalkenyl, heteroaryl or heterocyclyl; and m is an integer in the range 0 to 8, inclusive.

The term “alkyl” refers to a radical of a saturated straight or branched chain hydrocarbon group of, for example, 1-20 carbon atoms, or 1-12, 1-10, or 1-6 carbon atoms.

The term “alkenyl” refers to a radical of an unsaturated straight or branched chain hydrocarbon group of, for example, 2-20 carbon atoms, or 2-12, 2-10, or 2-6 carbon atoms, having at least one carbon-carbon double bond.

The term “alkynyl” refers to a radical of an unsaturated straight or branched chain hydrocarbon group of, for example, 2-20 carbon atoms, or 2-12, 2-10, or 2-6 carbon atoms, having at least one carbon-carbon triple bond.

The term “aliphatic” includes linear, branched, and cyclic alkanes, alkenes, or alkynes. In certain embodiments, aliphatic groups in the present invention are linear, branched or cyclic and have from 1 to about 20 carbon atoms.

The term “aralkyl” includes alkyl groups substituted with an aryl group or a heteroaryl group.

The term “heteroatom” includes an atom of any element other than carbon or hydrogen. Illustrative heteroatoms include boron, nitrogen, oxygen, phosphorus, sulfur and selenium, and alternatively oxygen, nitrogen or sulfur.

The term “halo” or “halogen” includes —F, —Cl, —Br, — or —I.

The term “perfluoro” refers to a hydrocarbon wherein all of the hydrogen atoms have been replaced with fluorine atoms. For example, —CF₃ is a perfluorinated methyl group.

The term “aryl” refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “heteroaryl” refers to a mono-, bi-, or multi-cyclic, aromatic ring system containing one, two, or three heteroatoms such as nitrogen, oxygen, and sulfur. Examples include pyrrole, furan, thiophene, imidazole, oxazole, thiazole, triazole, pyrazole, pyridine, pyrazine, pyridazine and pyrimidine, and the like. Heteroaryls can also be fused to non-aromatic rings.

The terms “heterocycle,” “heterocyclyl,” or “heterocyclic” refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkyl, alkenyl, alkynyl, aldehyde, alkylthio, alkanoyl, alkoxy, alkoxycarbonyl, amido, amino, aminothiocarbonyl, aryl, arylcarbonyl, arylthio, carboxy, cyano, cycloalkyl, cycloalkylcarbonyl, ester, ether, halogen, heterocyclyl, heterocyclylcarbonyl, hydroxy, ketone, oxo, nitro, sulfonate, sulfonyl, and thiol.

Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl. Heterocycles also include bridged bicyclic groups where a monocyclic heterocyclic group can be bridged by an alkylene group.

The heterocyclic or heteroaryl ring may be can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The terms “polycyclyl” and “polycyclic group” include structures with two or more rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms, e.g., three or more atoms are common to both rings, are termed “bridged” rings. Each of the rings of the polycycle may be substituted with such substituents as described above can be substituted with groups selected from alkyl, alkenyl, alkynyl, alkanoyl, alkoxy, alkoxy, alkylthio, amino, amido, aryl, aralkyl, azide, carbonyl, carboxy, cyano, cycloalkyl, ester, ether, halogen, haloalkyl, heterocyclyl, hydroxy, imino, ketone, nitro, perfluoroalkyl, phosphonate, phosphinate, silyl ether, sulfonamido, sulfonate, sulfonyl, and sulfhydryl.

The term “carbocycle” includes an aromatic or non-aromatic ring in which each atom of the ring is carbon.

The terms “amine” and “amino” include both unsubstituted and substituted amines, e.g., a moiety that may be represented by the general formulas:

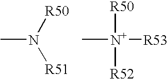

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and includes a moiety that may be represented by the general formula:

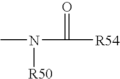

wherein R50 is as defined above, and R54 represents a hydrogen, an alkyl, an alkenyl or —(CH₂)ₘ—R61, where m and R61 are as defined above.

The term “amido” refers to an amino-substituted carbonyl and includes a moiety that may be represented by the general formula:

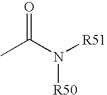

wherein R50 and R51 are as defined above. Certain embodiments of the amide in the present invention will not include imides which may be unstable.

The term “alkylthio” includes an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” includes such moieties as may be represented by the general formulas:

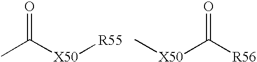

wherein X50 is a bond or represents an oxygen or a sulfur, and R55 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiocarbonyl” group. Where X50 is a sulfur and R55 or R56 is not hydrogen, the formula represents a “thioester.” Where X50 is a sulfur and R55 is hydrogen, the formula represents a “thiocarboxylic acid.” Where X50 is a sulfur and R56 is hydrogen, the formula represents a “thioformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” include an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” includes a moiety that may be represented by the general formula:

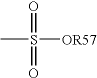

in which R57 is an electron pair, hydrogen, alkyl, cycloalkyl, or aryl.

The term “sulfate” includes a moiety that may be represented by the general formula:

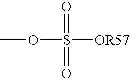

in which R57 is as defined above.

The term “sulfonamido” is art-recognized and includes a moiety that may be represented by the general formula:

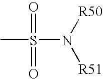

in which R50 and R51 are as defined above.

The term “sulfonyl” includes a moiety that may be represented by the general formula:

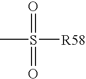

in which R58 is one of the following: hydrogen, alkyl, alkenyl, alkynyl, cycloalkyl, heterocyclyl, aryl or heteroaryl.

The term “sulfoxido” includes a moiety that may be represented by the general formula:

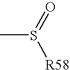

in which R58 is defined above.

The term “optionally substituted” or “substituted” is contemplated to include all permissible substituents of organic compounds. For example, substituted refers to a chemical group, such as alkyl, cycloalkyl, aryl, heteroaryl and the like, wherein one or more hydrogen atoms may be replaced with a substituent such as halogen, azide, alkyl, aralkyl, alkenyl, alklynyl, cycloalkyl, hydroxy, alkoxy, amino, amido, nitro, cyano, sulfhydryl, imino, phosphonate, phosphinate, carbonyl, carboxyl, silyl, ether, alkylthio, sulfonyl, sulfonamido, ketone, aldehyde, ester, heterocyclyl, aromatic or heteroaromatic moieties, perfluoroalkyl (e.g., —CF₃), acyl, and the like, or any of the substituents of the preceding paragraphs or any of those substituents either attached directly or by suitable linkers. The linkers are typically short chains of 1-3 atoms containing any combination of —C—, —C(O)—, —NH—, —S—, —S(O)—, —O—, —C(O)O— or —S(O)—. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The definition of each expression, e.g., alkyl, m, n, etc., when it occurs more than once in any structure, is intended to be independent of its definition elsewhere in the same structure unless otherwise indicated expressly or by the context.

The terms triflyl, tosyl, mesyl, and nonaflyl are art-recognized and refer to trifluoromethanesulfonyl, ρ-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, ρ-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art recognized and represent methyl, ethyl, phenyl, trifluoromethanesulfonyl, nonafluorobutanesulfonyl, ρ-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

The phrase “protecting group” includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2^{nd} ed., Wiley, New York, (1991). The phrase “hydroxyl-protecting group” includes those groups intended to protect a hydroxyl group against undesirable reactions during synthetic procedures and includes, for example, benzyl or other suitable esters or ethers groups known in the art. The aforementioned protecting groups may be present in the compounds of the invention, and are not limited to use only during synthesis of the compounds of the invention. Thus, the presence of a protecting group is not intended to suggest that said group must be removed. For example, the compounds of the present invention may contain an ether group, such as a methoxymethyl ether, which is a known hydroxyl protecting group.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

### Exemplary Substrates

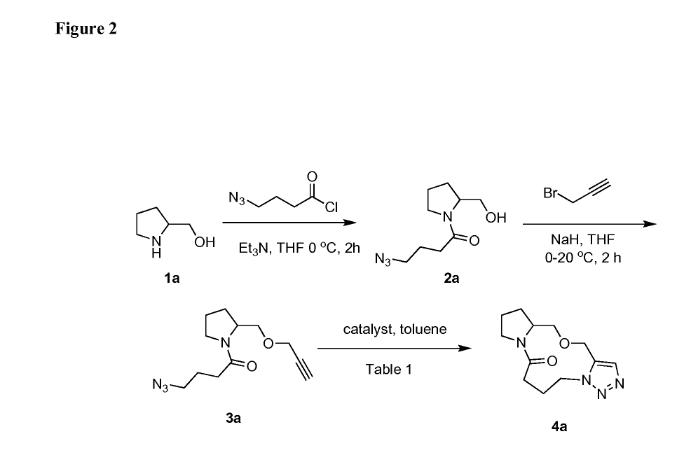

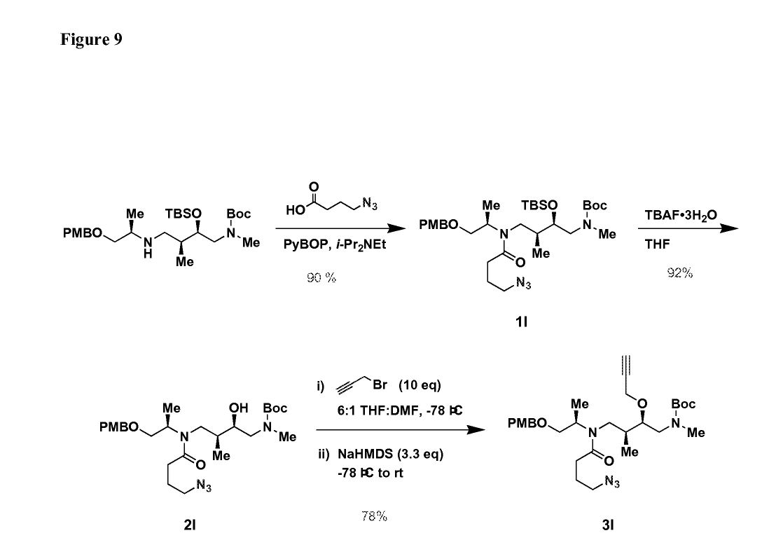

To explore the substrate scope of the divergent pairing strategy depicted in FIG. 1, various alkynyl azides embedded within different structural frameworks were synthesized (FIG. 2). The first pairing partner was provided by coupling an amino alcohol (1a) to an azido acyl chloride, resulting in the amide (2a). The second requisite functional group, the alkyne, was added via propargylation of the alcohol (3a). This general synthesis was applied to a variety of substrates. However, the linear (3e-g) and cyclohexyl substrates (3j-k) displayed bisacylation and protection of the alcohol as a silyl ether was necessary to avoid ester formation. Furthermore, FIG. 9 depicts the synthesis of substrate 3l, which is functionally and stereochemically more complex than substrates 3a-3k. Acylation of the amine with an azido acid to form compound 1l may alternatively be completed in the presence of DIEA in CH₂Cl₂ at about room temperature (not shown). Subsequent TBAF-mediated TBS deprotection of the resulting amide (1l) gave rise to alcohol 2l. Introduction of the alkyne component, however, proved difficult. Propargylation of alcohol 2l under standard conditions using NaH in DMF resulted in incomplete reaction even with a large excess of propargyl bromide. Biphasic propargylation conditions using aqueous NaOH in CH₂Cl₂ with a phase transfer catalyst also proved relatively ineffective. Further optimization led to the use of KHMDS or NaHMDS in a mixed solvent system (THF:DMF=about 6:1 or about 3:1) to affect the propargylation in high yield.

The methods of the present invention are widely applicable. Linear and pyrrolidine scaffolds are good substrates. Additionally, in certain embodiments, it can be envisioned that the macrocycles formed by the present invention are macrocyclic peptides.

### Exemplary Catalysts

Ruthenium

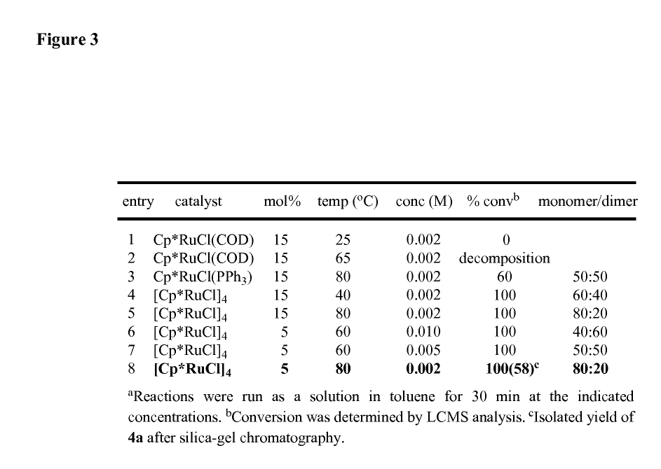

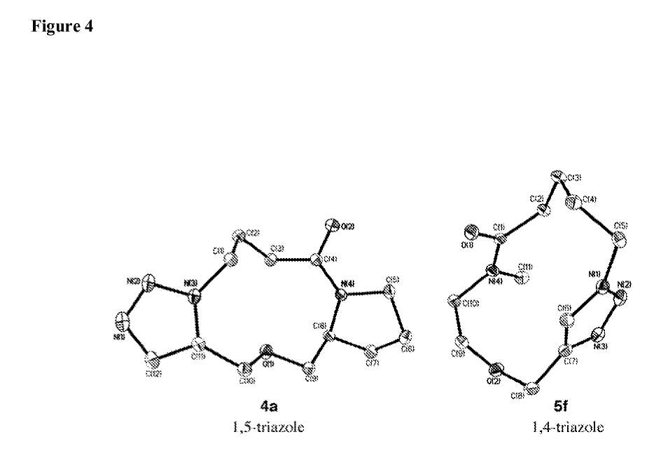

Ruthenium(II)-catalyzed formation of 1,5-triazoles was investigated. When Cp*RuCl (COD) (FIG. 3, entries 1 and 2) was employed as the catalyst, the reaction yielded no product even at elevated temperatures, most likely due to catalyst thermal instability. More encouraging results were acheived with Cp*RuCl (PPh₃) (FIG. 3, entry 3), however, 50% dimer formation was observed and purification of the desired product proved difficult due to the presence of phosphine oxide. Improved results were obtained with the [Cp*RuCl]₄ catalyst (FIG. 3, entries 4-8) which yielded improved monomer to dimer ratios (entries 5 and 8) and a more straightforward purification. Using 5% [Cp*RuCl]₄ (FIG. 3, entries 6-8) it was found that higher temperatures (80° C.) and lower concentrations (0.002 M) (entry 8) led to optimal monomer to dimer ratios. With this protocol in hand, the desired 1,5-macrocyclic triazole 4a in 58% isolated yield was obtained. The structure was confirmed to be the 1,5-regioisomer via X-ray crystallography (FIG. 4).

The macrocyclization of substrate 3l and its diastereomers was also investigated. During preliminary catalyst evaluation, [Cp*RuCl]₄ was identified as an ideal catalyst. The Ru-catalyzed reactions were reliable and could be routinely performed to yield about 5 g to about 10 g of the macrocycle triazoles.

Copper

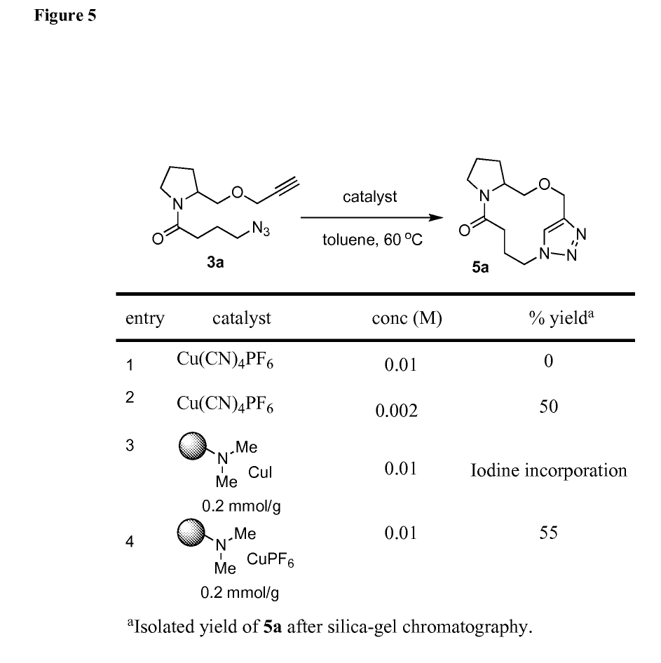

Copper-catalyzed formation of 1,4-triazoles was also investigated. The catalyst Cu(CN)₄PF₆ was utilized at 0.01 M in toluene at 60° C.; this reaction resulted in complete consumption of starting material but no desired product (FIG. 5, entry 1). It was determined this was due to the intermolecular formation of dimers and oligomers; therefore, the concentration of the reaction was decreased to favor the intramolecular reaction. Gratifyingly, by changing the reaction concentration from 0.01 M to 0.002 M the 1,4-triazole 5a was obtained in 50% yield (FIG. 5, entry 2). However, such dilute conditions are typically not preferred for reactions on a larger scale.

A recent publication by Girard and coworkers showcased the utility of CuI loaded on Amberlyst resin as a catalyst for an intermolecular Huisgen cycloaddition. Girard, C.; et al. Org. Lett. 2006, 8, 1689. Remarkably, we discovered that this technology could be used to facilitate pseudodilution and suppress dimer formation in the intramolecular Huisgen reaction. First, the conditions reported by Girard were used (Amberlyst-21 and CuI at 0.2 mmol/g loading) (FIG. 5, entry 3). The monomeric 1,4-triazole was obtained, however there was evidence of iodine incorporation. While this observation is not uncommon in the use of solution phase CuI, it was not reported by Girard with the solid-phase catalyst. In light of this problem, the protocol was applied to the generation of a CuPF₆ Amberlyst. The component CuPF₆ (as opposed to CuBr) was chosen due to its high solubility in CH₃CN, the solvent used in the preparation of the solid-supported copper reagents. Using this catalyst, a 5-fold increase in reaction concentration (0.2 mmol/g loading) was achieved, along with a slight increase in yield for the desired product (FIG. 5, entry 4).

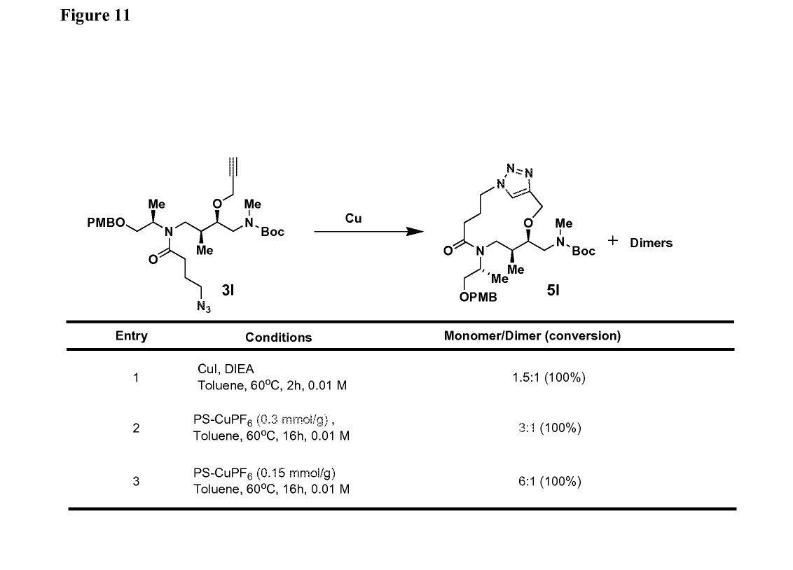

Complementary to the work depicted in FIG. 5, the effect of the Cu source on the specificity of the cyclization was investigated. The results are depicted in FIG. 11. Here, again, PS—CuPF₆ at low catalyst loading provided a desirable outcome (FIG. 11, entry 3). It was observed that the polymer-bound catalyst suppressed dimerization according to the effects of pseudodilution.

The macrocyclization of substrate 3l and its diastereomers was also examined. In this case as well, the polymer-bound catalyst was successful.

### Various Reaction Considerations

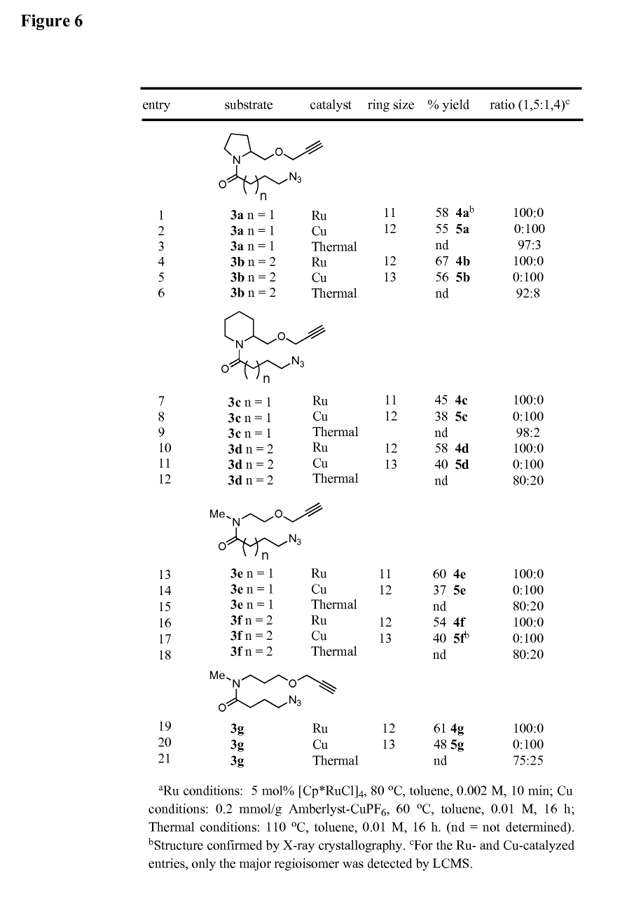

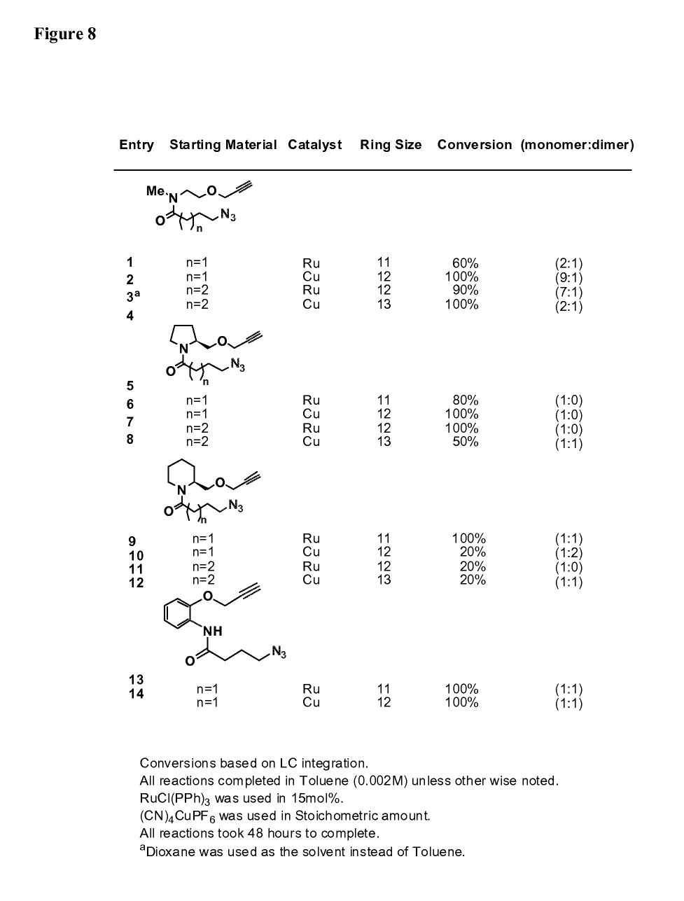

With these conditions in hand, the effect of substrate specificity, conformation, and ring size on these metal-catalyzed macrocyclizations was investigated. The results of these experiments are shown in FIGS. 8 and 6.

Substrate Conformation and Ring Size

FIG. 8 depicts results obtained by using preliminary Ru and Cu catalysts. As the table shows, an undesirable amount of the dimer was formed in many of the reactions.

Similar systems were examined utilizing different catalysts, as shown in FIG. 6. The examination of the pyrrolidine ring system containing an additional methylene group in the azido acid side chain (FIG. 6, entries 4-6) showed that the isolated yields for the formation of the 12- and 13-membered rings were slightly higher than that of the 11- and 12-membered rings, a trend which is consistent with thermodynamic arguments.

Since the pyrrolidine substrates (3a-b) assume a pseudo axial position, the piperidine azido alkynes (3c-d) were synthesized in order to examine the effect of a true axial substituent on ring closure. The resulting yields of the cycloaddition were slightly lower than that of their pyrrolidine counterparts (FIG. 6, entries 7-12). The drop in yield is most likely due to the difficulty of the cycloaddition. It is possible that the axial substituent places the alkyne and the azide further apart and leads to greater dimer and oligomer formation.

Three linear substrates (3e-g) were then tested to see if the rigidity imparted by the pyrrolidine ring was facilitating macrocyclization. The results of the metal-catalyzed cyclizations were very similar to that of the pyrrolidine substrate; however, the inherent bias in the system, determined by the thermal reaction, seemed to be less specific. When exposed to the thermal conditions the pyrrolidine substrates formed the 1,5-triazole products almost exclusively; whereas, the linear substrates gave a 4:1 ratio of the 1,5- to 1,4-triazoles (FIG. 6, entries 3 and 6 vs. 15 and 18). Substrates derived from 1,2-amino alcohols (3e-f) were also compared to substrates derived from 1,3-amino alcohols (3g) to determine if the position of the oxygen in the macrocyclic ring had any effect on the cycloaddition. The 1,3-amino alcohols (FIG. 6, entries 19 and 20) were only slightly higher in yield than their 1,2-amino alcohol counterparts (entries 16 and 17) and their thermal ratios were quite similar (entries 18 and 21). From these observations, it would not seem that the position of the alcohol has a dramatic effect on the macrocyclization. An X-ray crystal structure was obtained for one of the 1,4-triazoles (5f, FIG. 4).

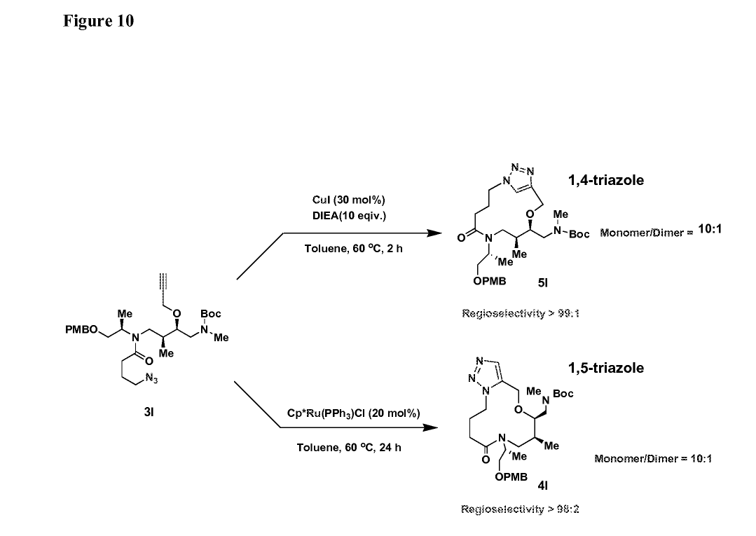

Furthermore, complex substrate 3l was cyclized by both methods, as depicted in FIG. 10, with an excellent monomer:dimer ratio (10:1) in both cases, and regioselectivities greater than 98:2. By this method, divergent pairing can be used to synthesize a library containing both a primary OH for loading onto a solid phase and one diversity site. Implementing the n and n+1 concept will double the library size while providing valuable structure-activity relationship information.

Solid-Phase Catalysts

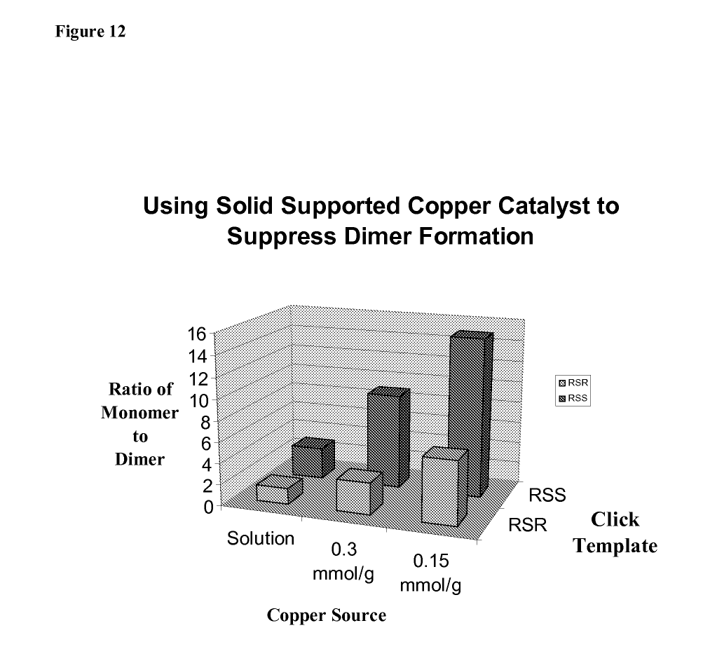

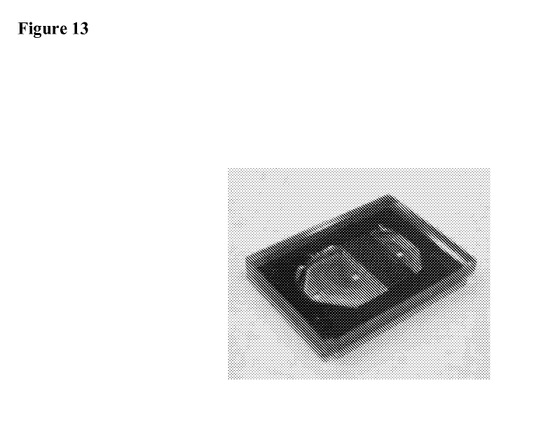

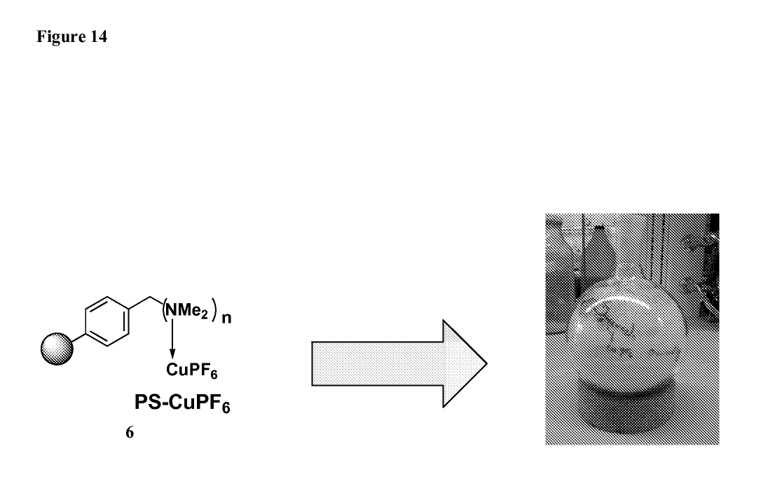

Additionally, the success of polymer-bound catalysts in the methods of the present invention is noteworthy. Data supporting this contention are supported in FIG. 12. As mentioned previously, the use of a polymer-bound catalyst suppresses dimerization. These particular methods have many advantages including: reaction monitoring, ease of loading, catalyst recyclability, and use in a flow reactor. Specifically, flow reactors may be used to increase the efficiency of the solid-phase reactions. A flow reactor for use with the methods of the present invention is depicted in FIG. 13. An exemplary solid-supported copper catalyst is shown in FIG. 14 (“PS—CuPF₆” or “PS—N(CH₃)₂CuPF₆” or 6, where PS is polystyrene); upon loading of the copper catalyst, the resin turns green.

Stereochemistry

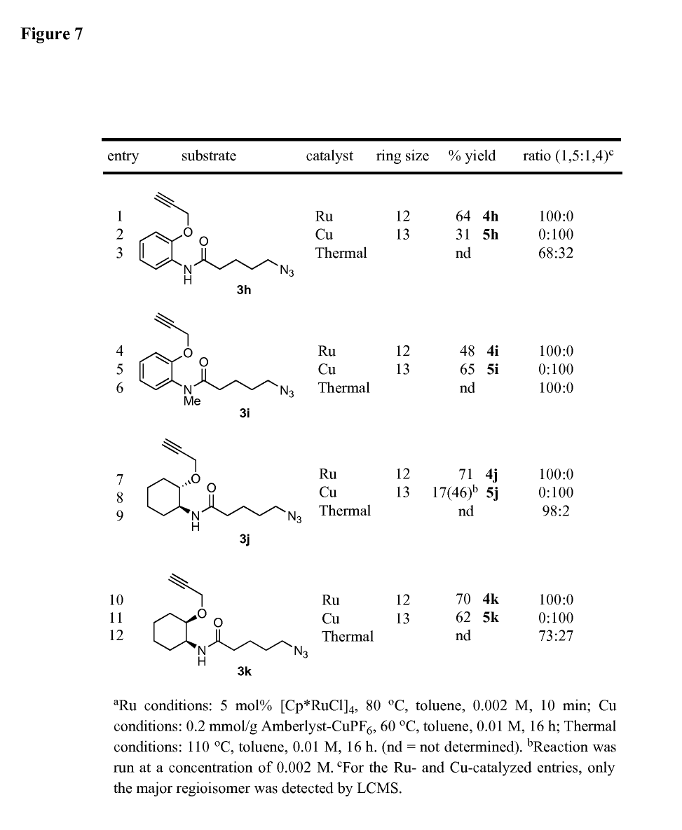

In addition to conformational effects, stereochemical effects on the macrocyclic triazole ring formation were also examined. The results of these experiments are shown in FIG. 7. The first system examined was the planar 2-aminophenol derivatives (FIG. 7, entries 1-6). To better understand the system, both the primary (FIG. 7, entries 1-3) and secondary amides (entries 4-6) were studied, however, no trend was observed. Both of these substrates gave moderate yields in all cases except for the copper-catalyzed primary amide (FIG. 7, entry 2). Next, a saturated system of the cis- and trans-cyclohexyl amino alcohol compounds (FIG. 7, entries 7-12) was examined. Surprisingly, the cis or trans configuration had little effect for the ruthenium-catalyzed reaction (FIG. 7, entries 7 and 10); however, the trans system showed a remarkable loss in yield for the copper-catalyzed case (FIG. 7, entry 8). Optimization of this reaction for this substrate was effected by a 5-fold dilution, leading to a significant increase in yield, from 17% to 46% (FIG. 7, entry 8).

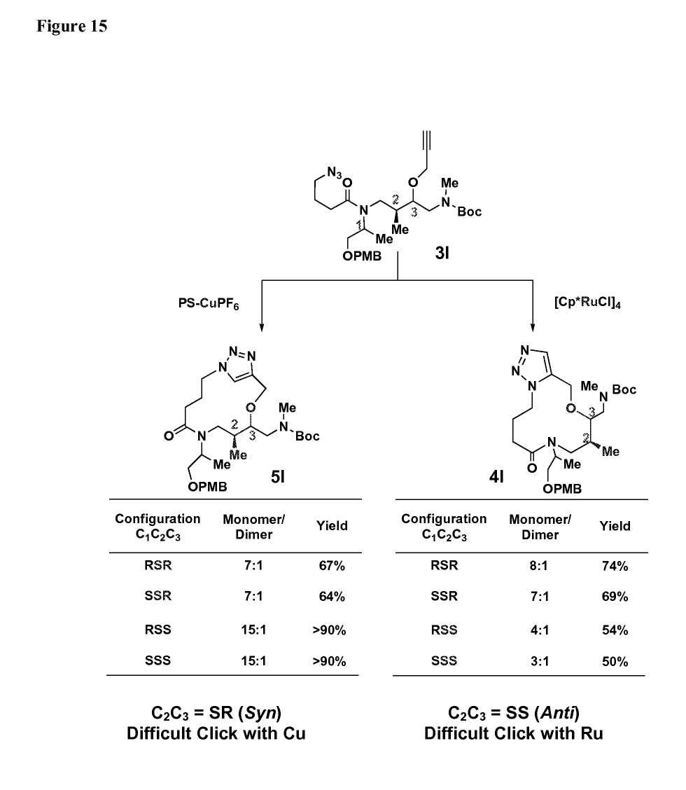

The success of exemplary macrocyclizations of the present invention was investigated for a substrate containing a more complex stereochemical environment (3l and its diastereomers, FIG. 15). Various stereoisomers of compounds 5l and 4l were synthesized, and the yields compared. As can be seen from the table in FIG. 15, the copper-catalyzed reaction is more successful when the C₂C₃ stereochemistry is SS (the anti-aldol derived substrates). These reactions may use, for example 0.5 equivalents of PS—CuPF₆, and be run in toluene at about 55° C. Alternatively, substrates containing SR C₂C₃ stereochemistry (the syn-aldol derived substrates) are more readily cyclized with a Ru catalyst. In the case of the Ru-catalyzed reaction depicted in FIG. 15, the Ru tetramer was used in place of the Cp*CuCl (PPh₃) to avoid the formation of PPh₃O, which was difficult to separate from the product. The [Cp*RuCl]₄ may be used in about 5 mol % and the reaction may be run in toluene at about 70° C. Decomposition was observed when Cp*RuCl COD was used. Interestingly, with respect to stereochemistry, the reactivity of the Cu-catalyzed cyclization is the reverse of what was observed for the Ru-catalyzed reaction.

### Post-Cyclization Modifications

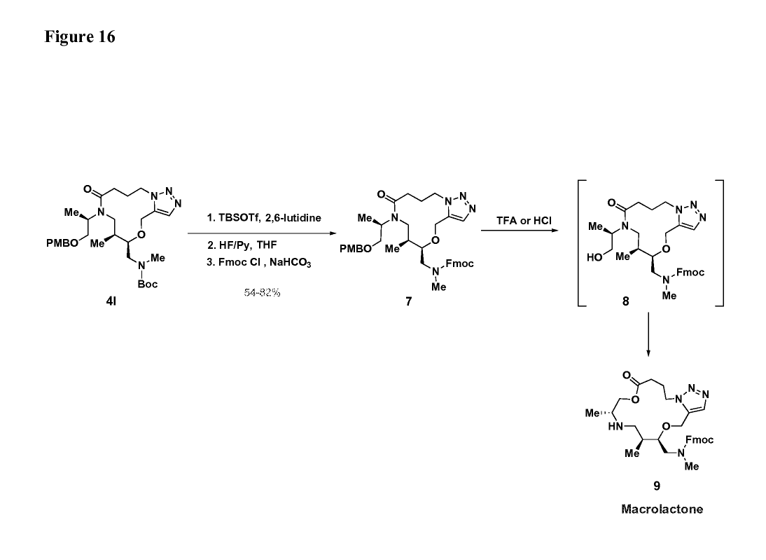

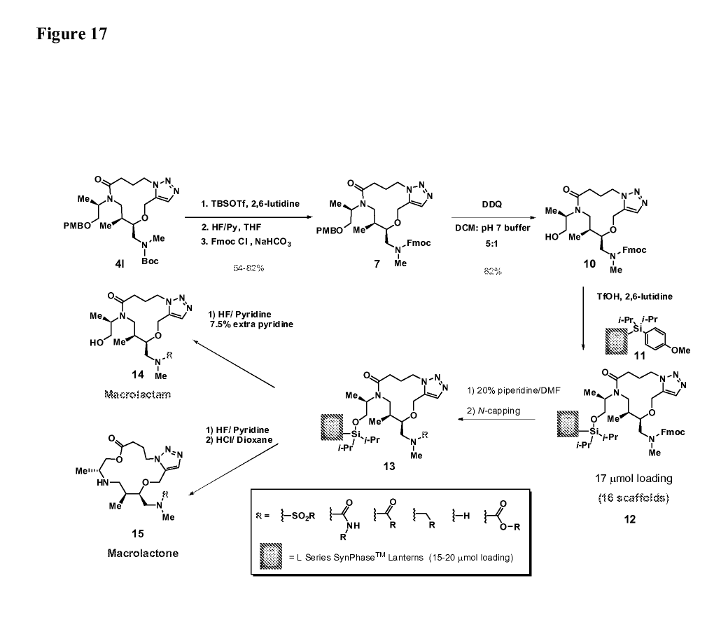

FIGS. 16 and 17 depict various post-cyclization modifications of an exemplary macrocycle (4l). The synthesis of a 15-membered macrolactone from a 12-membered macrolactam is depicted in FIG. 16. This reaction has also been accomplished using a solid support (FIG. 17). In both cases, initial attempts to remove the Boc and PMB protecting groups simultaneously under acidic (HCl or TFA) conditions led to inconsistent results. Addition of t-butyl cation was observed under TFA-mediated Boc removal. Surprisingly, this side reaction could not be suppressed by the addition of various scavengers. In light of these difficulties, an alternate deprotection sequence was pursued. Gratifyingly, TBSOTf could be employed for the chemoselective removal of the Boc group to afford the desired amine, proceeding through the silyl carbamate intermediate. Protection of the resulting amine as the Fmoc carbamate (7) proceeded smoothly. Finally, PMB removal could be achieved oxidatively usign DDQ (FIG. 17) to afford 10 in good yield and high purity.

The feasibility of executing reactions on a solid-phase scaffold was investigated (FIG. 17). Loading onto solid-support was achieved via activation of silicon-functionalized Lanterns with TfOH followed by reaction with the core (about 1.2 equivalents) in the presence of 2,6-lutidine to provide an average loading level of about 15 μmol/Lantern or about 17 μmol/Lantern. A representative selection of solid-phase transformations were explored for the introductions of appendage diversity. The Fmoc protecting group is removed under standard conditions (20% piperidine in DMF), yielding the secondary amine which is suitable for reaction with various electrophiles including sulfonyl chlorides, isocyanates, acids, and aldehydes. Following N-capping at the amine, cleavage from the solid support was achieved by treatment with 15% HF/pyridine to afford the desired product. Purity of the crude final products exceeded about 95% as judged by UPCL analysis at 210 nm.

### Exemplary Methods

In certain embodiments, the invention relates to a method of forming a triazole according to Scheme 1:

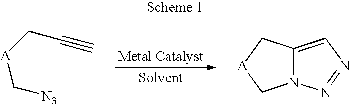

wherein, independently for each occurrence,

A is -(a)ₘ-;

metal catalyst consists essentially of at least one ligand and Ru;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned methods, wherein -(a)ₘ- comprises an amide.

In certain embodiments, the invention relates to any one of the aforementioned methods, wherein -(a)ₘ- comprises an amino acid.

In certain embodiments, the invention relates to any one of the aforementioned methods, wherein m is 7, 8, or 9.

In certain embodiments, the invention relates to any one of the aforementioned methods, wherein m is 7.

In certain embodiments, the invention relates to any one of the aforementioned methods, wherein m is 8.

In certain embodiments, the invention relates to any one of the aforementioned methods, wherein A is —O—CR₂—CR₂—NR—C(═O)—CR₂—CR₂—.

In certain embodiments, the invention relates to any one of the aforementioned methods, wherein A is —O-phenyl-NR—C(═O)—CR₂—CR₂—CR₂—.

In certain embodiments, the invention relates to any one of the aforementioned methods, wherein A is —O—CR₂—CR₂—NR—C(═O)—CR₂—CR₂—CR₂—.

In certain embodiments, the invention relates to any one of the aforementioned methods, wherein A is —O—CR₂—CR₂—CR₂—NR—C(═O)—CR₂—CR₂—.

In certain embodiments, the invention relates to any one of the aforementioned methods, wherein the metal catalyst is [Cp*RuCl]₄, Cp*RuCl (COD), or Cp*RuCl (PPh₃).

In certain embodiments, the invention relates to any one of the aforementioned methods, wherein the metal catalyst is [Cp*RuCl]₄.

In certain embodiments, the invention relates to a method of forming a triazole according to Scheme 2:

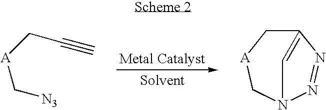

wherein, independently for each occurrence,

A is -(a)ₘ-;

metal catalyst consists essentially of at least one ligand and Cu;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned methods, wherein -(a)ₘ- comprises an amide.

In certain embodiments, the invention relates to any one of the aforementioned methods, wherein -(a)ₘ- comprises an amino acid.

In certain embodiments, the invention relates to any one of the aforementioned methods, wherein m is 7, 8, or 9.

In certain embodiments, the invention relates to any one of the aforementioned methods, wherein m is 7.

In certain embodiments, the invention relates to any one of the aforementioned methods, wherein m is 8.

In certain embodiments, the invention relates to any one of the aforementioned methods, wherein A is —O—CR₂—CR₂—NR—C(═O)—CR₂—CR₂—.

In certain embodiments, the invention relates to any one of the aforementioned methods, wherein A is —O-phenyl-NR—C(═O)—CR₂—CR₂—CR₂—.

In certain embodiments, the invention relates to any one of the aforementioned methods, wherein A is —O—CR₂—CR₂—NR—C(═O)—CR₂—CR₂—CR₂—.

In certain embodiments, the invention relates to any one of the aforementioned methods, wherein A is —O—CR₂—CR₂—CR₂—NR—C(═O)—CR₂—CR₂—.

In certain embodiments, the invention relates to any one of the aforementioned methods, wherein the metal catalyst is Cu (CH₃CN)₂PF₆, (CN)₄CuPF₆, CuI, PS—N(CH₃)₂CuI, or PS—N(CH₃)₂CuPF₆.

In certain embodiments, the invention relates to any one of the aforementioned methods, wherein the metal catalyst is PS—N(CH₃)₂CuPF₆.

In certain embodiments, the invention relates to a method of forming a triazole according to Scheme 3:

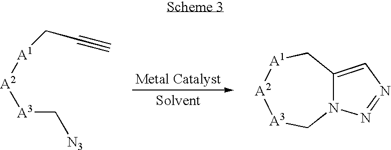

wherein, independently for each occurrence,

A¹ is —(CR₂)ₙ—, —(CR₂)ₙ—O—, —O—(CR₂)ₙ—, —O—(CR₂)ₙ—O—, —(CR₂)ₙ—NR—;

A² is —NR—C(═O)—, —C(═O)—NR—, or

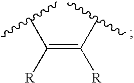

A³ is —(CR₂)ₙ—;

metal catalyst consists essentially of at least one ligand and Ru;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned methods, wherein A¹ is —(CR₂)ₙ— or —(CR₂)ₙ—O—.

In certain embodiments, the invention relates to any one of the aforementioned methods, wherein A¹ is —(CR₂)₃—O—.

In certain embodiments, the invention relates to any one of the aforementioned methods, wherein A¹ is —(CR₂)₂—O— or —(CR₂)₃—O—.

In certain embodiments, the invention relates to any one of the aforementioned methods, wherein A² is —NR—C(═O)— or —C(═O)—NR—.

In certain embodiments, the invention relates to any one of the aforementioned methods, wherein A³ is —(CR₂)₂— or —(CR₂)₃—.

In certain embodiments, the invention relates to any one of the aforementioned methods, wherein -A¹-A²-A³- is —O—(CR₂)₂—NR—C(═O)—(CR₂)₂—.

In certain embodiments, the invention relates to any one of the aforementioned methods, wherein -A¹-A²-A³- is —O—(CR₂)₂—NR—C(═O)—(CR₂)₃—.

In certain embodiments, the invention relates to any one of the aforementioned methods, wherein -A¹-A²-A³- is —O—(CR₂)₃—NR—C(═O)—(CR₂)₂—.

In certain embodiments, the invention relates to any one of the aforementioned methods, wherein -A¹-A²-A³- is selected from the group consisting of

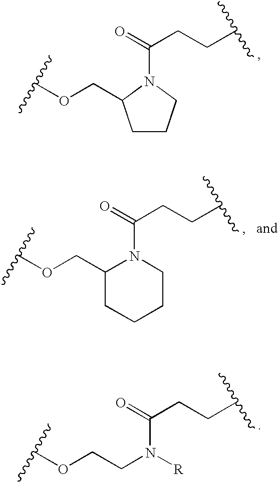

In certain embodiments, the invention relates to any one of the aforementioned methods, wherein -A¹-A²-A³- is selected from the group consisting of

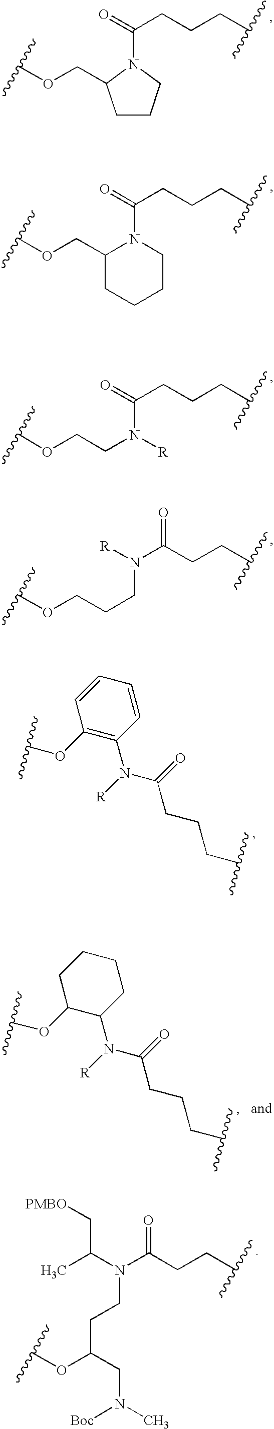

In certain embodiments, the invention relates to any one of the aforementioned methods, wherein the metal catalyst is [Cp*RuCl]₄, CP*RuCl (COD), or Cp*RuCl (PPh₃).

In certain embodiments, the invention relates to any one of the aforementioned methods, wherein the metal catalyst is [Cp*RuCl]₄.

In certain embodiments, the invention relates to a method of forming a triazole according to Scheme 4:

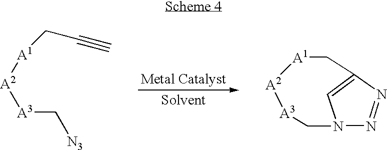

wherein, independently for each occurrence,

A¹ is —(CR₂)ₙ—, —(CR₂)ₙ—O—, —O—(CR₂)ₙ—O—, —(CR₂)ₙ—NR—;

A² is —NR—C(O)—, —C(O)—NR—, or

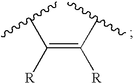

A³ is —(CR₂)ₙ—;

metal catalyst consists essentially of at least one ligand and Cu;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned methods, wherein A¹ is —(CR₂)ₙ— or —(CR₂)ₙ—O—.

In certain embodiments, the invention relates to any one of the aforementioned methods, wherein A¹ is —(CR₂)ₙ—O—.

In certain embodiments, the invention relates to any one of the aforementioned methods, wherein A¹ is —(CR₂)₂—O— or —(CR₂)₃—O—.

In certain embodiments, the invention relates to any one of the aforementioned methods, wherein A² is —NR—C(O)— or —C(O)—NR—.

In certain embodiments, the invention relates to any one of the aforementioned methods, wherein A³ is —(CR₂)₂— or —(CR₂)₃—.

In certain embodiments, the invention relates to any one of the aforementioned methods, wherein -A¹-A²-A³- is —O—(CR₂)₂—NR—C(═O)—(CR₂)₂—.

In certain embodiments, the invention relates to any one of the aforementioned methods, wherein -A¹-A²-A³- is —O—(CR₂)₂—NR—C(═O)—(CR₂)₃—.

In certain embodiments, the invention relates to any one of the aforementioned methods, wherein -A¹-A²-A³- is —O—(CR₂)₃—NR—C(═O)—(CR₂)₂—.

In certain embodiments, the invention relates to any one of the aforementioned methods, wherein -A¹-A²-A³- is selected from the group consisting of

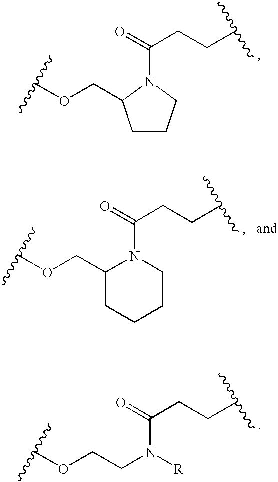

In certain embodiments, the invention relates to any one of the aforementioned methods, wherein -A¹-A²-A³- is selected from the group consisting of

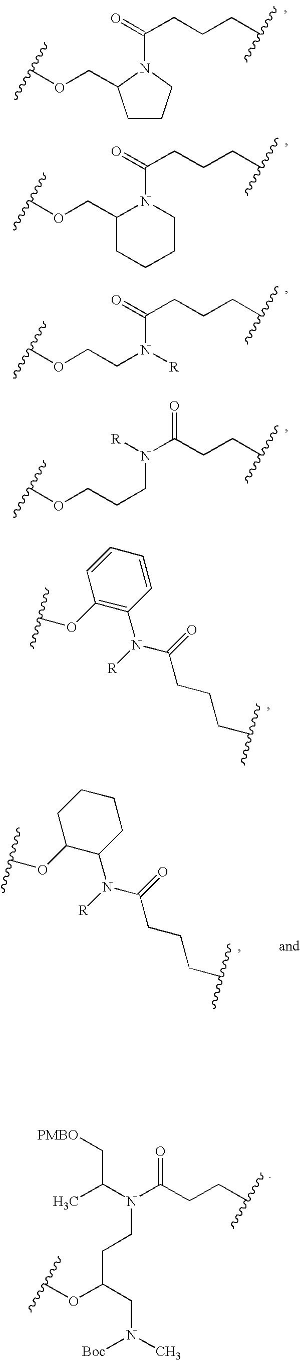

In certain embodiments, the invention relates to any one of the aforementioned methods, wherein the metal catalyst is Cu (CH₃CN)₂PF₆, (CN)₄CuPF₆, CuI, PS—N(CH₃)₂CuI, or PS—N(CH₃)₂CuPF₆.

In certain embodiments, the invention relates to any one of the aforementioned methods, wherein the metal catalyst is PS—N(CH₃)₂CuPF₆.

In certain embodiments, the invention relates to any one of the aforementioned methods, wherein the triazole is obtained in an isolated yield of greater than about 50%.

In certain embodiments, the invention relates to any one of the aforementioned methods, wherein the triazole is obtained in an isolated yield of greater than about 60%.

In certain embodiments, the invention relates to any one of the aforementioned methods, wherein the triazole is obtained in an isolated yield of greater than about 70%.

In certain embodiments, the invention relates to any one of the aforementioned methods, wherein the triazole is obtained in an isolated yield of greater than about 80%.

In certain embodiments, the invention relates to any one of the aforementioned methods, wherein the triazole is obtained in an isolated yield of greater than about 90%.

In certain embodiments, the invention relates to any one of the aforementioned methods, wherein the solvent is selected from the group consisting of toluene, xylene, methyl-t-butyl ether, diisopropyl ether, and 2-propanol.

In certain embodiments, the invention relates to any one of the aforementioned methods, wherein the solvent is toluene.

### Reaction Conditions

The reactions of the present invention may be performed under a wide range of conditions, though it will be understood that the solvents and temperature ranges recited herein are not limitative and only correspond to a preferred mode of the process of the invention.

In general, it will be desirable that reactions are run using mild conditions which will not adversely effect the substrate, the catalyst, or the product. For example, the reaction temperature influences the speed of the reaction, as well as the stability of the reactants, products, and catalyst. The reactions will usually be run at temperatures in the range of −78° C. to 100° C.

In general, the cyclization reactions of the present invention are carried out in a liquid reaction medium. The reactions may be run without addition of solvent. Alternatively, the reactions may be run in an inert solvent, preferably one in which the reaction ingredients, including the catalyst, are substantially soluble. Suitable solvents include ethers such as diethyl ether, 1,2-dimethoxyethane, diglyme, methyl-t-butyl ether, tetrahydrofuran, diisopropyl ether, and the like; halogenated solvents such as chloroform, dichloromethane, dichloroethane, chlorobenzene, and the like; aliphatic or aromatic hydrocarbon solvents such as benzene, toluene, hexane, pentane, xylene, and the like; esters and ketones such as ethyl acetate, acetone, and 2-butanone; polar aprotic solvents such as acetonitrile, dimethylsulfoxide, dimethylformamide and the like; alcohols, such as 2-propanol, and the like; or combinations of two or more solvents. Furthermore, in certain embodiments it may be advantageous to employ a solvent which is not inert to the substrate under the conditions employed, e.g., use of ethanol as a solvent when ethanol is the desired nucleophile.

The invention also contemplates reaction in a biphasic mixture of solvents, in an emulsion or suspension, or reaction in a lipid vesicle or bilayer. In certain embodiments, it may be preferred to perform the catalyzed reactions in the solid phase.

In some preferred embodiments, the reaction may be carried out under an atmosphere of a reactive gas. The partial pressure of the reactive gas may be from 0.1 to 1000 atmospheres, more preferably from 0.5 to 100 atm, and most preferably from about 1 to about 10 atm.

In certain embodiments it is preferable to perform the reactions under an inert atmosphere of a gas such as nitrogen or argon.

The processes of the present invention can be conducted in continuous, semi-continuous or batch fashion and may involve a liquid recycle and/or gas recycle operation as desired. The processes of this invention are preferably conducted in batch fashion. Likewise, the manner or order of addition of the reaction ingredients, catalyst and solvent are also not critical and may be accomplished in any conventional fashion.

The reaction can be conducted in a single reaction zone or in a plurality of reaction zones, in series or in parallel or it may be conducted batchwise or continuously in an elongated tubular zone or series of such zones. The materials of construction employed should be inert to the starting materials during the reaction and the fabrication of the equipment should be able to withstand the reaction temperatures and pressures. Means to introduce and/or adjust the quantity of starting materials or ingredients introduced batchwise or continuously into the reaction zone during the course of the reaction can be conveniently utilized in the processes especially to maintain the desired molar ratio of the starting materials. The reaction steps may be effected by the incremental addition of one of the starting materials to the other. Also, the reaction steps can be combined by the joint addition of the starting materials to the optically active metal-ligand complex catalyst. When complete conversion is not desired or not obtainable, the starting materials can be separated from the product and then recycled back into the reaction zone.

The processes may be conducted in either glass lined, stainless steel or similar type reaction equipment. The reaction zone may be fitted with one or more internal and/or external heat exchanger(s) in order to control undue temperature fluctuations, or to prevent any possible “runaway” reaction temperatures.

Furthermore, as mentioned above, the catalyst can be immobilized or incorporated into a polymer or other insoluble matrix by, for example, derivatization with one or more of substituents of the ligand. The immobilized ligands can be complexed with the desired metal to form the metallocatalyst. The catalyst, particularly an “aged” catalyst, is easily recovered after the reaction as, for instance, by filtration or centrifugation.

### Exemplary Compounds

In certain embodiments, the invention relates to a compound of formula I

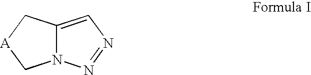

or a pharmaceutically acceptable salt thereof,
wherein, independently for each occurrence,

A is -(a)ₘ-;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned compounds, wherein -(a)ₘ- comprises an amide.

In certain embodiments, the invention relates to any one of the aforementioned compounds, wherein -(a)ₘ- comprises an amino acid.

In certain embodiments, the invention relates to any one of the aforementioned compounds, wherein m is 7, 8, or 9.

In certain embodiments, the invention relates to any one of the aforementioned compounds, wherein m is 7.

In certain embodiments, the invention relates to any one of the aforementioned compounds, wherein m is 8.

In certain embodiments, the invention relates to any one of the aforementioned compounds, wherein A is —O—CR₂—CR₂—NR—C(═O)—CR₂—CR₂—.

In certain embodiments, the invention relates to any one of the aforementioned compounds, wherein A is —O-phenyl-NR—C(═O)—CR₂—CR₂—CR₂—.

In certain embodiments, the invention relates to any one of the aforementioned compounds, wherein A is —O—CR₂—CR₂—NR—C(═O)—CR₂—CR₂—CR₂—.

In certain embodiments, the invention relates to any one of the aforementioned compounds, wherein A is —O—CR₂—CR₂—CR₂—NR—C(═O)—CR₂—CR₂—.

In certain embodiments, the invention relates to a compound of formula II

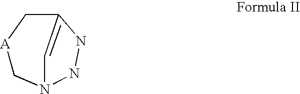

or a pharmaceutically acceptable salt thereof,
wherein, independently for each occurrence,

A is -(a)ₘ-;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned compounds, wherein -(a)ₘ- comprises an amide.

In certain embodiments, the invention relates to any one of the aforementioned compounds, wherein -(a)ₘ- comprises an amino acid.

In certain embodiments, the invention relates to any one of the aforementioned compounds, wherein m is 7, 8, or 9.

In certain embodiments, the invention relates to any one of the aforementioned compounds, wherein m is 7.

In certain embodiments, the invention relates to any one of the aforementioned compounds, wherein m is 8.

In certain embodiments, the invention relates to any one of the aforementioned compounds, wherein A is —O—CR₂—CR₂—NR—C(═O)—CR₂—CR₂—.

In certain embodiments, the invention relates to any one of the aforementioned compounds, wherein A is —O-phenyl-NR—C(═O)—CR₂—CR₂—CR₂—.

In certain embodiments, the invention relates to any one of the aforementioned compounds, wherein A is —O—CR₂—CR₂—NR—C(═O)—CR₂—CR₂—CR₂—.

In certain embodiments, the invention relates to any one of the aforementioned compounds, wherein A is —O—CR₂—CR₂—CR₂—NR—C(═O)—CR₂—CR₂—.

In certain embodiments, the invention relates to a compound of formula III

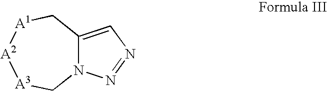

or a pharmaceutically acceptable salt thereof,
wherein, independently for each occurrence,

A¹ is —(CR₂)ₙ—, —(CR₂)ₙ—O—, —O—(CR₂)ₙ—, —O—(CR₂)ₙ—O—, —(CR₂)ₙ—NR—;

A² is —NR—C(═O)—, —C(═O)—NR—, or

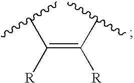

A³ is —(CR₂)ₙ—;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned compounds, wherein A¹ is —(CR₂)ₙ— or —(CR₂)ₙ—O—.

In certain embodiments, the invention relates to any one of the aforementioned compounds, wherein A¹ is —(CR₂)ₙ—O—.

In certain embodiments, the invention relates to any one of the aforementioned compounds, wherein A¹ is —(CR₂)₂—O— or —(CR₂)₃—O—.

In certain embodiments, the invention relates to any one of the aforementioned compounds, wherein A² is —NR—C(═O)— or —C(═O)—NR—.

In certain embodiments, the invention relates to any one of the aforementioned compounds, wherein A³ is —(CR₂)₂— or —(CR₂)₃—.

In certain embodiments, the invention relates to any one of the aforementioned compounds, wherein -A¹-A²-A³- is —O—(CR₂)₂—NR—C(═O)—(CR₂)₂—.

In certain embodiments, the invention relates to any one of the aforementioned compounds, wherein -A¹-A²-A³- is —O—(CR₂)₂—NR—C(═O)—(CR₂)₃—.

In certain embodiments, the invention relates to any one of the aforementioned compounds, wherein -A¹-A²-A³- is —O—(CR₂)₃—NR—C(═O)—(CR₂)₂—.

In certain embodiments, the invention relates to any one of the aforementioned compounds, wherein -A¹-A²-A³- is selected from the group consisting of

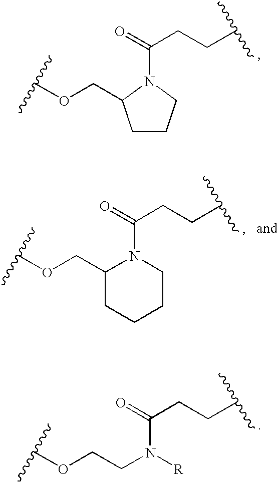

In certain embodiments, the invention relates to any one of the aforementioned compounds, wherein -A¹-A²-A³- is selected from the group consisting of

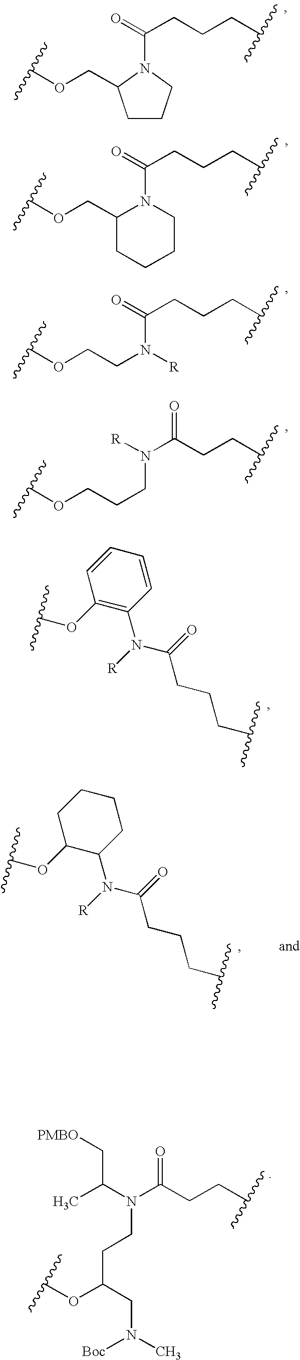

In certain embodiments, the invention relates to a compound of formula IV

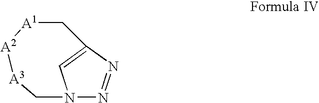

or a pharmaceutically acceptable salt thereof,
wherein, independently for each occurrence,

A¹ is —(CR₂)ₙ—, (CR₂)ₙ—O—, —O—(CR₂)ₙ—, —O—(CR₂)ₙ—O—, —(CR₂)ₙ—NR—;

A² is —NR—C(O)—, —C(O)—NR—, or

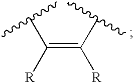

A³ is —(CR₂)ₙ—;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned compounds, wherein A¹ is —(CR₂)ₙ— or —(CR₂)ₙ—O—.

In certain embodiments, the invention relates to any one of the aforementioned compounds, wherein A¹ is —(CR₂)ₙ—O—.

In certain embodiments, the invention relates to any one of the aforementioned compounds, wherein A¹ is —(CR₂)₂—O— or —(CR₂)₃—O—.

In certain embodiments, the invention relates to any one of the aforementioned compounds, wherein A² is —NR—C(O)— or —C(O)—NR—.

In certain embodiments, the invention relates to any one of the aforementioned compounds, wherein A³ is —(CR₂)₂— or —(CR₂)₃—.

In certain embodiments, the invention relates to any one of the aforementioned compounds, wherein -A¹-A²-A³- is —O—(CR₂)₂—NR—C(═O)—(CR₂)₂—.

In certain embodiments, the invention relates to any one of the aforementioned compounds, wherein -A¹-A²-A³- is —O—(CR₂)₂—NR—C(═O)—(CR₂)₃—.

In certain embodiments, the invention relates to any one of the aforementioned compounds, wherein -A¹-A²-A³- is —O—(CR₂)₃—NR—C(═O)—(CR₂)₂—.

In certain embodiments, the invention relates to any one of the aforementioned compounds, wherein -A¹-A²-A³- is selected from the group consisting of

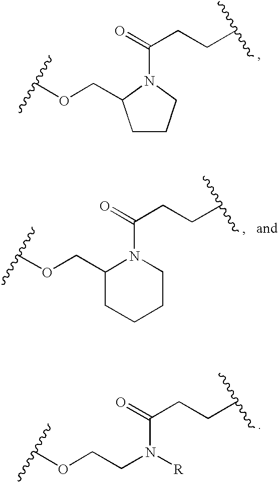

In certain embodiments, the invention relates to any one of the aforementioned compounds, wherein -A¹-A²-A³- is selected from the group consisting of

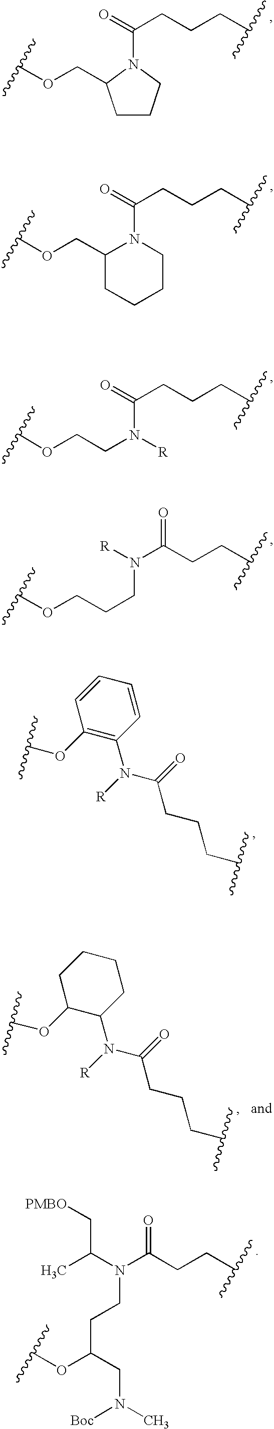

In certain embodiments, the invention relates to a compound, or a pharmaceutically acceptable salt thereof, selected from the group consisting of

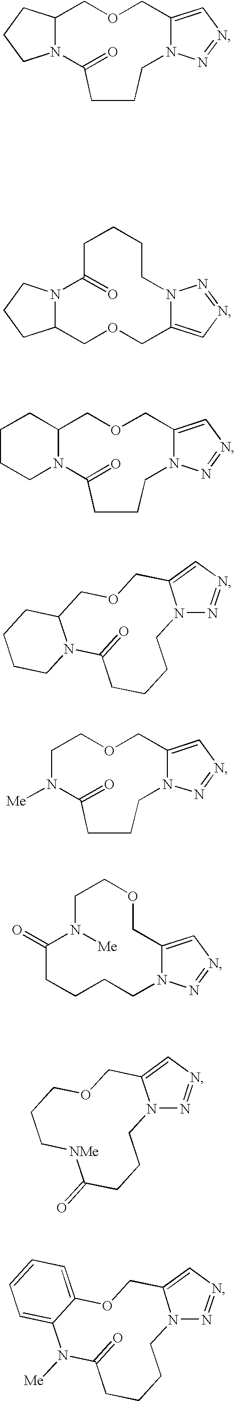

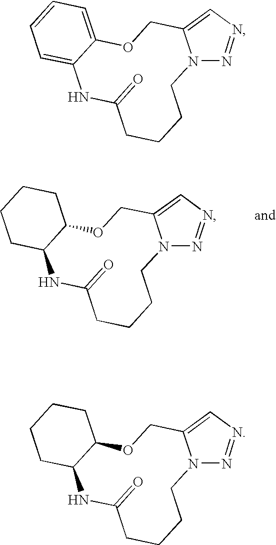

In certain embodiments, the invention relates to a compound, or a pharmaceutically acceptable salt thereof, selected from the group consisting of

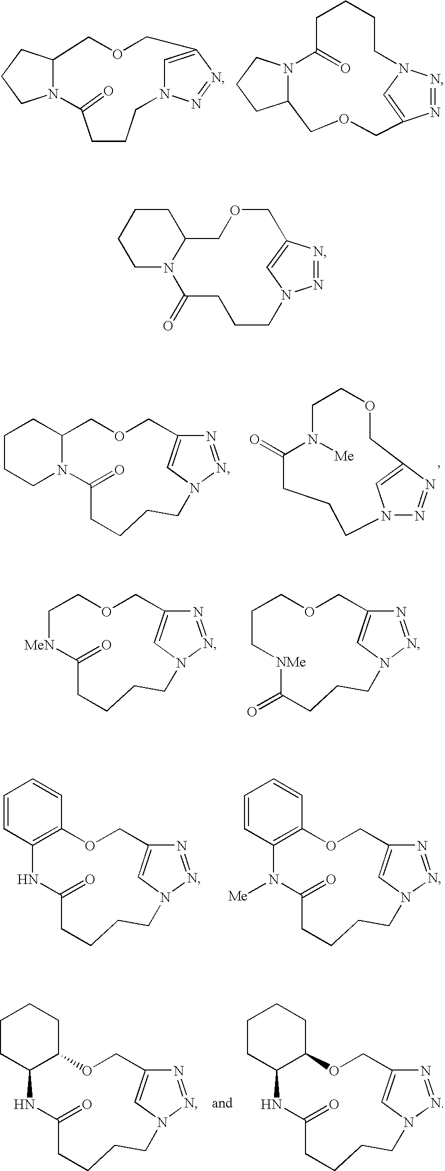

In certain embodiments, the invention relates to a compound of formula V

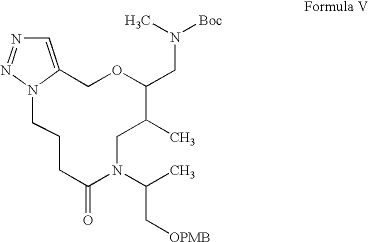

or a pharmaceutically acceptable salt thereof.

In certain embodiments, the invention relates to a compound of formula VI

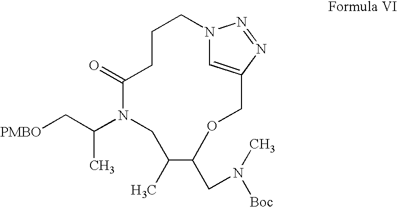

or a pharmaceutically acceptable salt thereof.

### Exemplary Libraries

In certain embodiments, the invention relates to a library comprising a plurality of compounds of formula I and a plurality of compounds of formula II

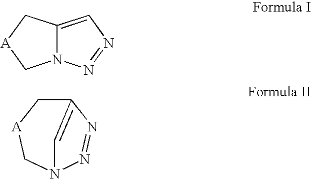

or a pharmaceutically acceptable salt thereof,
wherein, independently for each occurrence,

A is -(a)ₘ-;

a represents —O—, —NR—, —C(═O)—, —CR₂—, —S—, —RP(═O)—, —S(═O)—, —SO₂—, or phenyl;

m is 6, 7, 8, 9, 10, 11, or 12; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to a library comprising a plurality of compounds of formula III and a plurality of compounds of formula IV

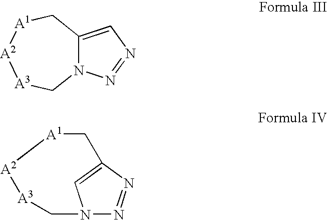

or a pharmaceutically acceptable salt thereof,
wherein, independently for each occurrence,

A¹ is —(CR₂)ₙ—, —(CR₂)ₙ—O—, —O—(CR₂)ₙ—, —O—(CR₂)ₙ—O—, —(CR₂)ₙ—NR—;

A² is —NR—C(═O)—, —C(═O)—NR—, or

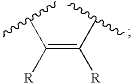

A³ is —(CR₂)ₙ—;

n is 1, 2, 3, 4, or 5; and

R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

In certain embodiments, the invention relates to any one of the aforementioned libraries, wherein said compounds are covalently linked to a solid support. In certain embodiments, the invention relates to any one of the aforementioned libraries, wherein said solid support is a plurality of polystyrene beads. In certain embodiments, the invention relates to any one of the aforementioned libraries, wherein said solid support is a plurality of polystyrene lanterns. In certain embodiments, the invention relates to any one of the aforementioned libraries, wherein said solid support is a plurality of polyamide lanterns. In certain embodiments, the invention relates to any one of the aforementioned libraries, wherein said solid support is a microtiter plate. In certain embodiments, the invention relates to any one of the aforementioned libraries, wherein said solid support is a 96-well microtiter plate.

### Combinatorial Libraries

The subject reactions readily lend themselves to the creation of combinatorial libraries of compounds for the screening of pharmaceutical, agrochemical or other biological or medically-related activity or material-related qualities. A combinatorial library for the purposes of the present invention is a mixture of chemically related compounds which may be screened together for a desired property; said libraries may be in solution or covalently linked to a solid support. The preparation of many related compounds in a single reaction greatly reduces and simplifies the number of screening processes which need to be carried out. Screening for the appropriate biological, pharmaceutical, agrochemical or physical property may be done by conventional methods.

Diversity in a library can be created at a variety of different levels. For instance, the substrate aryl groups used in a combinatorial approach can be diverse in terms of the core aryl moiety, e.g., a variegation in terms of the ring structure, and/or can be varied with respect to the other substituents.

A variety of techniques are available in the art for generating combinatorial libraries of small organic molecules. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; Chen et al. (1994) JACS 116:2661: Kerr et al. (1993) JACS 115:252; PCT publications WO92/10092, WO93/09668 and WO91/07087; and the Lerner et al. PCT publication WO93/20242). Accordingly, a variety of libraries on the order of about 16 to 1,000,000 or more diversomers can be synthesized and screened for a particular activity or property.

In an exemplary embodiment, a library of substituted diversomers can be synthesized using the subject reactions adapted to the techniques described in the Still et al. PCT publication WO 94/08051, e.g., being linked to a polymer bead by a hydrolyzable or photolyzable group, e.g., located at one of the positions of substrate. According to the Still et al. technique, the library is synthesized on a set of beads, each bead including a set of tags identifying the particular diversomer on that bead. In one embodiment, the beads can be dispersed on the surface of a permeable membrane, and the diversomers released from the beads by lysis of the bead linker. The diversomer from each bead will diffuse across the membrane to an assay zone, where it will interact with an enzyme assay. Detailed descriptions of a number of combinatorial methodologies are provided below.

### Direct Characterization

A growing trend in the field of combinatorial chemistry is to exploit the sensitivity of techniques such as mass spectrometry (MS), e.g., which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

### Multipin Synthesis

The libraries of the subject method can take the multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (c.f., Bray et al. (1990) Tetrahedron Lett 31:5811-5814; Valerio et al. (1991) Anal Biochem 197:168-177; Bray et al. (1991) Tetrahedron Lett 32:6163-6166).

### Divide-Couple-Recombine

In yet another embodiment, a variegated library of compounds can be provided on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

### Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

### Encoded Combinatorial Libraries

In yet another embodiment, the subject method utilizes a compound library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with Sequenceable Bio-Oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115:2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2) Non-Sequenceable Tagging: Binary Encoding

An alternative form of encoding the test compound library employs a set of non-sequencable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded.

### EXEMPLIFICATION

### General Considerations

All reactions were carried out under an atmosphere of N₂. All glassware was flame dried prior to use. Copper iodide, tetrakis(acetonitrile) copper hexafluorophospate, and chloro(pentamethylcyclopentadienyl)ruthenium (II) tetramer were purchased from Strem and used as received. All amino alcohols were used as received. Toluene, dichloromethane, and tetrahydrofuran were all commercial anhydrous and degassed with N₂ previous to use. NMR spectra were recorded on a Bruker 300 (300 MHz ¹H, 75 MHz ¹³C), Varian UNITY INOVA 500 (500 MHz ¹H, 125 MHz ¹³C) spectrometers. Proton and Carbon chemical shifts are reported in ppm (δ) referenced to the NMR solvent. Data are reported as follows: chemical shifts, multiplicity (br=broad singlet, s=singlet, d=doublet, t=triplet, q=quartet, p=pentet, m=multiplet; coupling constant(s) in Hz; integration). Unless otherwise indicated NMR data were collected at 25° C. Infrared spectra were obtained on a Perkin-Elmer Model 2000 FT-IR spectrometer and are reported in cm⁻¹. Flash chromatography was performed using 40-60 μm Silica Gel (60 A mesh) on a Teledyne Isco Combiflash Rf. Tandem Liquid Chromotography/Mass Spectrometry (LCMS) was performed on a Waters 2795 separations module and 3100 mass detector. Analytical thin layer chromatography was performed on EM Reagent 0.25 mm silica gel 60-F plates. Visualization was accomplished with UV light and aqueous potassium permanganate (KMnO₄) stain followed by heating. High resolution mass spectra were obtained at the MIT Mass Spectrometry Facility. X-ray crystallographic analysis was performed at the MIT X-ray crystallographic Laboratory by Dr. Peter Muller.

### Example 1

### Synthesis of Various Silyloxy Azide Compounds

### General Procedure C

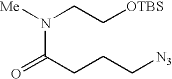

(1e) 4-azido-N-(2-(tert-butyldimethylsilyloxy)ethyl)-N-methylbutanamide: A round bottom flask with stir bar under a blanket of N₂ was charged with 2-(tert-butyldimethylsilyloxy)-N-methylethanamine (1.54 g, 8.13 mmol), PyBOP (4.23 g, 8.13 mmol) and dry dichloromethane (60 mL). Hunig's Base (4.26 mL, 24.4 mmol) was then added to the mixture slowly and it was cooled to 0° C. before 4-azidobutanoic acid (1.05 g, 8.13 mmol) was added as a solution in dry dichloromethane (10 mL) via syringe. After the addition was complete, the reaction was allowed to warm to RT and stirred overnight. Upon, determination that the reaction was complete via TLC and LCMS, the reaction was quenched using 20 mL water and extracted (3×50 mL ethyl acetate). Combined organic extracts were dried over MgSO₄ and concentrated in vacuo. The resulting crude mixture was dissolved in 150 mL diethylether and solids (PyBOP impurities) were filtered off. The filter cake was washed with diethylether and the resulting solution concentrated in vacuo. The compound was purified using column chromatography (30% ethyl acetate in hexanes) to provide the title compound as a yellow oil (2.00 g, 82% yield). IR (cm⁻¹) 2930, 2858, 2242, 2096, 1737, 1639, 1471, 1403, 1361, 1255, 1104, 1005, 909, 835, 811. ¹H NMR (500 MHz, CDCl₃) δ3.68 (dt, J=5.4, 8.7 Hz, 1H), 3.63 (s, 1H), 3.40 (dt, J=5.4, 24.8 Hz, 1H), 3.35-3.27 (m, 2H), 3.03 (s, 2H), 2.88 (s, 1H), 2.43 (t, J=7.2 Hz, 1H), 2.35 (dt, J=7.2, 10.5 Hz, 2H), 1.91-1.80 (m, 2H), 0.83 (s, 9H), −0.01 (d, J=4.7 Hz, 6H). ¹³C NMR (1.1:1 rotamer ratio, asterisk denotes minor rotamer peaks, 125 MHz, CDCl₃) δ 172.3*, 171.7, 61.8*, 60.6, 51.9, 51.2*, 51.1, 50.9*, 50.7*, 37.6, 33.9*, 31.0, 30.2, 29.8*, 26.0, 25.9*, 24.6*, 24.4, 18.3*, 18.2, −5.3. HRMS (ESI) calcd for [M+Na]+: C₁₃H₂₈N₄NaO₂Si: 323.1874. Found: 323.1877.

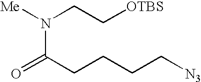

(1f) 4-azido-N-(2-(tert-butyldimethylsilyloxy)ethyl)-N-methylpentamide: This compound was prepared using General Procedure C using Hunig's Base (12.6 mL, 72.0 mmol), 2-(tert-butyldimethylsilyloxy)-N-methylethanamine (5.00 g, 26.4 mmol), PyBOP (12.49 g, 24.00 mmol), and 4-azidopentanoic acid (3.44 g, 24.0 mmol) in dry dichloromethane (170 mL) to provide the title compound as a yellow oil (5.00 g, 66% yield). IR (cm⁻¹) 2929, 2857, 2238, 2093, 1636, 1471, 1402, 1361, 1253, 1104, 1006, 909, 835, 811, 776, 727. ¹H NMR (1.1:1 rotamer ratio, asterisk denotes minor rotamer peaks, 300 MHz, CDCl₃) δ 3.69 (dd, J=5.5, 12.4 Hz, 2H), 3.43*(t, J=5.4 Hz, 2H), 3.37 (t, J=5.4 Hz, 2H), 3.26 (m, 2H), 3.03 (s, 3H), 2.89*(s, 3H), 2.38*(t, J=7.0 Hz, 2H), 2.29 (t, J=7.0 Hz, 2H), 1.63 (m, 2H), 0.84 (s, 9H), 0*(s, 6H), −0.01 (s, 6H). ¹³C NMR (1.1:1 rotamer ratio, asterisk denotes minor rotamer peaks, 125 MHz, CDCl₃) δ 172.9*, 172.3, 61.8, 60.7*, 51.9, 51.5*, 51.4*, 50.9, 37.7*, 32.5, 28.8, 28.7*, 26.0,*25.9, 22.5, 22.2*, 18.3, 18.3*, −5.34, and -5.42. HRMS (ESI) calcd for [M+H]+: C₁₄H₃₁N₄O₂Si: 315.2211. Found: 315.2221.

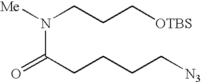

(1g) 5-azido-N-(3-(tert-butyldimethylsilyloxy)propyl)-N-methylpentanamide: This compound was prepared using General Procedure C using Hunig's Base (9.02 mL, 51.6 mmol), 3-(tert-butyldimethylsilyloxy)-N-methylpropan-1-amine (3.50 g, 17.2 mmol), PyBOP (9.85 g, 18.93 mmol), and 4-azidopentanoic acid (2.44 g, 18.9 mmol) in dry dichloromethane (120 mL) to provide the title compound as a yellow oil (4.23 g, 78% yield). IR (cm⁻¹) 3369, 2929, 2856, 2181, 2091, 1619, 1462, 1289, 1254, 1177, 1129, 1060, 946, 877, 833. ¹H NMR (1.1:1 rotamer ratio, asterisk denotes minor rotamer peaks, 500 MHz, CDCl₃) δ 3.57 (t, J=6.30 Hz, 1H), 3.56*(t, J=5.2 Hz, 2H), 3.34 (m, 4H), 2.95*(s, 3H), 2.86 (s, 3H), 2.40 (t, 1H, J=7.1 Hz), 2.33*(t, 1H, J=7.1 Hz), 1.87 (p, 2H, J=6.9 Hz) ppm 1.71-1.65*(m, 2H), 0.84 (s, 9H), 0.83*(s, 9H), 0.00 (s, 6H), −0.01*(s, 6H). ¹³C NMR (1.1:1 rotamer ratio, asterisk denotes minor rotamer peaks, 125 MHz, CDCl₃) δ 177.0, 176.8*, 66.1*, 64.9, 56.4, 51.8*, 50.7, 41.1, 38.7*, 36.7, 36.0*, 35.5*, 34.7, 31.3*, 31.3, 29.9, 29.7*, 23.7*, 23.6, 0.1*, 0.0. HRMS (ESI) calcd for [M+H]+: C₁₄H₃₁N₄O₂Si: 315.2211. Found: 315.2215.

### General Procedure J

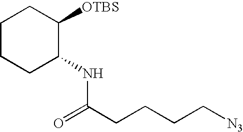

(1j) trans-5-azido-N-((trans)-2-(tert-butyldimethylsilyloxy)cyclohexyl)pentanamide:

A round bottom flask was charged with cis- and trans-2-(tert-butyldimethylsilyloxy)cyclohexanamine (12.0 g, 52.3 mmol), tetrahydrofuran (300 mL) and triethylamine (21.87 mL, 157 mmol). At 0° C., a solution of 4-azidopentanoic acid (8.45 g, 52.3 mmol) in tetrahydrofuran was added dropwise and the mixture was stirred for 1 hour at 0° C. then warmed to RT. The reaction was checked by TLC and determined to be complete. Then, the reaction quenched with sat. NH₄Cl and extracted in ethyl acetate (3×20 mL). Organic phase washed once with 0.5 M HCl and with brine, dried over MgSO₄, filtered and concentrated. The reaction was purified using column chromatography (0-100% ethyl acetate in hexanes) to provide the title compound (1.4 g of the trans, 62% yield of both isomers). IR (cm⁻¹) 3291, 2930, 2857, 2095, 1640, 1553, 1462, 1249, 1102, 878. ¹H NMR (500 MHz, CDCl₃) δ 5.30 (br s, 1H, —NH), 3.64 (m, 1H), 3.41 (m, 1H), 3.29 (t, J=6.7 Hz, 2H), 2.17 (dt, J=2.0, 7.2 Hz, 2H), 2.09 (m, 1H), 1.82 (m, 1H), 1.75-1.67 (m, 3H), 1.66-1.59 (m, 2H), 1.59-1.52 (m, 1H), 1.37 (m, 2H), 1.25 (m, 1H), 1.13 (m, 1H), 0.87 (s, 9H), 0.06 (d, J=3.6, 6H). ¹³C NMR (75 MHz, CDCl₃) δ 171.7, 73.1, 51.1, 36.2, 34.1, 30.6, 28.4, 25.6, 23.8, 23.5, 22.7, 17.9, −4.2, −4.7. HRMS (ESI) calcd for [M+Na]⁺: C₁₇H₃₄N₄NaO₂Si: 377.2343. Found: 377.2343.

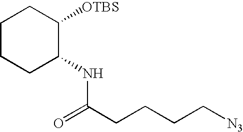

(1k) 5-azido-N-((cis)-2-(tert-butyldimethylsilyloxy)cyclohexyl)pentanamide: This compound was prepared using General Procedure J using cis- and trans-2-(tert-butyldimethylsilyloxy)cyclohexanamine (12.0 g, 52.3 mmol), 4-azidopentanoic acid (8.45 g, 52.3 mmol), and Et₃N (21.87 mL, 157 mmol) in tetrahydrofuran (300 mL). The reaction was purified using column chromatography (0-100% ethyl acetate in hexanes) to give the title compound in 10 g of the trans, 62% yield of both isomers. IR (cm⁻¹) 3323, 2931, 2856, 2094, 1641, 1541, 1462, 1251, 1131, 1022, 914, 837, 776, 674. ¹H NMR (500 MHz, CDCl₃) δ 5.59 (d, J=8.5 Hz, 1H), 3.91 (br s, 1H, —NH), 3.81 (m, 1H), 3.29 (t, J=6.7 Hz, 2H), 2.16 (t, J=7.3 Hz, 2H), 1.71 (m, 3H), 1.63 (m, 3H), 1.58-1.42 (m, 4H), 1.39-1.24 (m, 2H), 0.93 (s, 9H), 0.06 (d, J=9.0 Hz, 6H). ¹³C NMR (75 MHz, CDCl₃) δ 171.0, 69.6, 51.2, 50.9, 36.3, 32.5, 28.4, 27.2, 25.8, 24.5, 22.8, 19.1, 18.1, −4.4, −4.9. HRMS (ESI) calcd for [M+Na]⁺: C₁₇H₃₄N₄NaO₂Si. Found: 377.2343.

### Example 2

### Syntheses of Various Hydroxy Azide Compounds

### General Procedure A

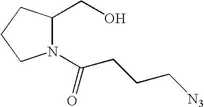

(2a) 4-azido-1-(2-(hydroxymethyl)pyrrolidin-1-yl)butan-1-one: A round bottom flask with a stir bar was charged with 4-azidobutanoic acid (2.02 g, 15.5 mmol) in dichloromethane (10 mL) and placed under N₂ atmosphere. Thionyl chloride (1.71 mL, 23.5 mmol) was added at RT and the resulting mixture was heated at 40° C. for 4 h. At this time the solution was cooled and the solvent removed in vacuo and the acid chloride was carried on without any purification. Then, a separate round bottom flask was charged with a stir bar and 2-methanol pyrrolidine (1.73 g 17.2 mmol), triethylamine (4.34 mL, 31.2 mmol) and tetrahydrofuran (140 mL). 4-Azidobutanoyl chloride (15.5 mmol) in tetrahydrofuran (28.0 mL) was added slowly to the mixture at 0° C. After 4 h, the reaction appeared complete by TLC and LCMS. The reaction was quenched with water, solvent removed in vacuo, and then extracted with ethyl acetate (3×100 mL). The reaction mixture was dried over MgSO₄ and concentrated in vacuo. The reaction was purified using column chromatography. (5% methanol in dichloromethane) (1.70 g, 52% yield). IR (cm⁻¹) 3378, 2952, 2875, 2091, 1612, 1437, 1346, 1281, 1253, 1047. ¹H NMR (300 MHz, CDCl₃) δ 4.68 (s, 1H), 4.25-4.09 (m, 1H), 3.72-3.40 (m, 4H), 3.37 (t, J=6.4 Hz, 2H), 2.38 (t, J=7.1 Hz, 2H), 2.09-1.78 (m, 5H), 1.66-1.50 (m, 1H). ¹³C NMR (125 MHz, CDCl₃) δ 173.4, 67.9, 61.7, 50.8, 48.5, 31.8, 28.2, 25.2, 24.2. HRMS (ESI) calcd for [M+Na]⁺: C₉H₁₆N₄NaO₂: 235.1166. Found: 235.1161.

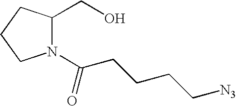

(2b) 4-azido-1-(2-(hydroxymethyl)pyrrolidin-1-yl)pentan-1-one: This compound was prepared using General Procedure A using 2-methanol pyrrolidine (1.57 g 15.5 mmol), 4-azidopentanoyl chloride (2.28 g, 14.11 mmol) and triethylamine (3.93 mL, 28.2 mmol) to provide the title compound as a yellow oil (2.75 g, 86% yield). IR (cm⁻¹) 3380, 2944, 2873, 2090, 1612, 1429, 1352, 1246, 1192, 1160, 1048, 899. ¹H NMR (4.5:1 rotamer ratio, asterisk denotes minor rotamer peaks, 500 MHz, CDCl₃) δ 4.93 (br s, 1H), 4.41*(br s, 1H), 3.96-3.87 (m, 1H), 3.75-3.69*(m, 1H), 3.34 (d, J=5.1 Hz, 1H), 3.30-3.18 (m, 3H), 3.07 (t, J=6.6 Hz, 2H), 2.20*(t, J=7.2 Hz, 2H), 2.10 (t, J=7.1 Hz, 2H), 1.81-1.69 (m, 1H), 1.69-1.60 (m, 1H), 1.51-1.43 (m, 3H), 1.43-1.37 (m, 3H). ¹³C NMR (125 MHz, CDCl₃) δ 173.9, 67.5, 61.4, 51.5, 48.3, 34.5, 28.7, 28.5, 24.6, 22.1. HRMS (ESI) calcd for [M+Na]⁺: C₁₀H₁₈N₄NaO₂: 249.1322. Found: 249.1317.

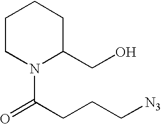

(2c) 4-azido-1-(2-(hydroxymethyl)piperidin-1-yl)butan-1-one: This compound was prepared using General Procedure A using piperidin-2-ylmethanol (1.10 g, 9.55 mmol), 4-azidobutanoyl chloride (2.20 g, 14.9 mmol) and triethylamine (3.02 g, 29.8 mmol) to provide the title compound as a yellow oil (1.15 g, 34% yield). IR (cm⁻¹) 3388, 2939, 2869, 2094, 1612, 1444, 1350, 1263, 1168, 1139, 1050, 1021, 895. ¹H NMR (1.3:1 rotamer ratio, asterisk denotes minor rotamer peaks, 500 MHz, CDCl₃) δ 4.50 (s, 1H), 4.27 (d, J=13.3 Hz, 1H), 3.84*(s, 1H), 3.73 (t, J=10.3 Hz, 1H), 3.57-3.28 (m, 2H), 3.25-3.05 (m, 2H), 2.91 (t, J=12.0 Hz, 1H), 2.41*(t, J=12.0, 1H), 2.33-2.12 (m, 2H), 1.67 (p, J=6.9 Mz, 2H), 1.56-1.03 (m, 8H). ¹³C NMR (1.3:1 rotamer ratio, asterisk denotes minor rotamer peaks, 125 MHz, CDCl₃) δ 172.5, 172.1*, 61.5, 60.6*, 54.7*, 51.2, 51.1, 50.9*, 41.9, 36.9*, 30.5, 30.3*, 26.3*, 25.8, 25.4*, 25.0, 24.8*, 24.6, 19.8*, 19.6. HRMS (ESI) calcd for [M+Na]⁺: C₁₀H₁₈N₄NaO₂: 249.1322. Found: 249.1327.

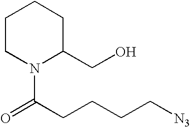

(2d) 5-azido-1-(2-(hydroxymethyl)piperidin-1-yl)pentan-1-one: This compound was prepared using General Procedure A using piperidin-2-ylmethanol (1.79 g, 15.5 mmol), 4-azidopentanoyl chloride (2.28 g, 14.1 mmol) and triethylamine (2.86 g, 28.2 mmol) to provide the title compound as a yellow oil (2.75 g, 81% yield). IR (cm⁻¹) 3381, 2938, 2868, 2092, 1611, 1438, 1264, 1050, 1021. ¹H NMR (500 MHz, CDCl₃) δ 4.55 (br s, 1H), 4.40 (s, 1H), 4.16 (s, 1H), 3.75 (s, 1H), 3.58 (s, 1H), 3.40-3.20 (m, 2H), 2.99 (d, J=4.9 Hz, 1H), 2.82 (t, J=11.9 Hz, 1H), 2.33 (t, J=12.1 Hz, 1H), 2.16 (s, 1H) 2.07 (s, 1H), 1.57-0.94 (m, 8H). ¹³C NMR (1.3:1 rotamer ratio, asterisk denotes minor rotamer peaks, 125 MHz, CDCl₃) δ 172.1, 60.1*, 59.9, 54.2, 50.7, 49.8*, 41.4*, 36.4, 32.6*, 32.2, 28.1, 28.0*, 25.7, 25.4*, 25.0, 24.3*, 22.1, 22.0*, 19.2, 18.9*. HRMS (ESI) calcd for [M+Na]⁺: C₁₁H₂₀N₄NaO₂: 263.1479. Found: 263.1490.

### General Procedure D

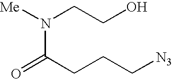

(2e) 4-azido-N-(2-hydroxyethyl)-N-methylbutanamide: A round bottom flask with stir bar was charged with 4-azido-N-(2-(tert-butyldimethylsilyloxy)ethyl)-N-methylbutanamide (2.44 g, 8.12 mmol) and purged with N₂. tetrahydrofuran (80 mL) was then added and the reaction flask was cooled to 0° C. Then, TBAF (16.2 mL, 16.2 mmol, 1 M in tetrahydrofuran) was added slowly to the reaction mixture. Once the addition was complete, the reaction was allowed to warm to RT. After 2 h, the reaction was deemed complete by TLC and LCMS analysis, quenched with 20 mL water and the tetrahydrofuran was removed in vacuo. The water layer was extracted (3×50 mL ethyl acetate) and the organic layer was washed with acetic acid pH 4 and the organic layer was dried over MgSO₄. The solvent was removed in vacuo and the compound was purified using column chromatography (0-100% ethyl acetate in hexanes) to provide the title compound as a yellow oil (1.00 g, 66% yield). IR (cm⁻¹) 3051, 2963, 2936, 2875, 2095, 1721, 1633, 1265, and 1074. ¹H NMR (1.4:1 rotamer ratio, asterisk denotes minor rotamer peaks, 500 MHz, CDCl₃) δ 3.74-3.67 (m, 2H), 3.49 (t, J=5.3 Hz, 2H), 3.41*(t, J=5.4 Hz, 2H), 3.37 (br s, 1H), 3.35-3.28 (m, 2H), 3.03 (s, 3H), 2.90*(s, 3H), 2.46*(t, J=7.2 Hz, 2H), 2.38 (t, J=7.2 Hz, 2H), 1.92-1.81 (m, 2H). ¹³C NMR (2.1:1 rotamer ratio, asterisk denotes minor rotamer peaks, 125 MHz, CDCl₃) δ 173.7, 172.8*, 61.3, 59.7*, 52.0*, 51.4, 51.1*, 51.0, 36.8, 33.8*, 30.2, 29.8*, 24.6*, 24.3. HRMS (ESI) calcd for [M+H]⁺: C₇H₁₅N₄O₂: 187.1190. Found: 187.1197.

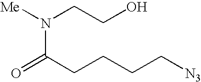

(2f) 4-azido-N-(2-hydroxyethyl)-N-methylpentamide: This compound was prepared using General Procedure D using 4-azido-N-(2-(tert-butyldimethylsilyloxy)ethyl)-N-methylpentamide (5.00 g, 15.9 mmol), TBAF (31.8 mL, 31.8 mmol) and tetrahydrofuran (100 mL) provide the title compound as a yellow oil (2.50 g, 79% yield). IR (cm⁻¹) 3376, 2937, 2873, 2091, 1740, 1618, 1460, 1403, 1355, 1246, 1129, 1050, 915, 860, 729, 644, 557, 469, 436, 421, and 403. ¹H NMR (2:1 rotamer ratio, asterisk denotes minor rotamer peaks, 500 MHz, CDCl₃) δ 3.67 (t, J=5.29 Hz, 2H), 3.47 (t, J=5.13 Hz, 2H), 3.38*(t, J=5.13 Hz, 2H), 3.23 (dd, J=6.95, 15.1 Hz, 2H), 3.07 (br s, 1H, OH), 3.00 (s, 3H), 2.87*(s, 3H), 2.38*(t, J=7.11 Hz, 2H), 2.30 (t, J=7.11 Hz, 2H), 1.78-1.72*(m, 4H), 1.68-1.52 (m, 4H). ¹³C NMR (2:1 rotamer ratio, asterisk denotes minor rotamer peaks, 125 MHz, CDCl₃) δ 174.42, 173.53*, 61.45, 59.73*, 52.13*, 51.46 (2C), 36.96*, 33.80, 33.10*, 32.55, 28.69, 22.69*, 22.45. HRMS (ESI) calcd for [M+Na]⁺: C₈H₁₆N₄NaO₂: 223.1166. Found: 223.1170.

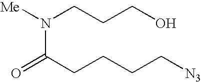

(2g) 5-azido-N-(3-(tert-butyldimethylsilyloxy)propyl)-N-methylpentanamide: This compound was prepared using General Procedure D using 5-azido-N-(3-(tert-butyldimethylsilyloxy)propyl)-N-methylpentanamide (4.00 g, 12.7 mmol), TBAF (19.1 mL, 19.1 mmol, 1.0 M in tetrahydrofuran) and tetrahydrofuran (127 mL) to provide the title compound as a yellow oil (2.45 g, 90% yield). IR (cm⁻¹) 3398, 2936, 2873, 2091, 1618, 1488, 1445, 1407, 1348, 1289, 1258, 1176, 1129, 1060, 946, 920, 862. ¹H NMR (5:1 rotamer ratio, asterisk denotes minor rotamer peaks, 500 MHz, CDCl₃) δ 3.59 (t, J=5.5 Hz, 2H)*, 3.47 (t, J=6.1 Hz, 2H), 3.42 (t, J=5.6 Hz 1H), 3.38 (t, J=7.5 Hz 2H)*, 3.33 (t, 2H, J=6.5 Hz), 2.94 (s, 3H), 2.89 (s, 3H)*, 2.42 (t, J=7 Hz, 2H)*, 2.39 (t, J=7.0 Hz, 2H), 1.90 (p, 1H, J=6.8 Hz), 1.74 (p, J=6.5 Hz, 2H)*, 1.64 (p, J=6.0 Hz, 2H). ¹³C NMR (5:1 rotamer ratio, asterisk denotes minor rotamer peaks, 125 MHz, CDCl₃) δ 173.3, 172.1*, 59.1*, 58.2, 51.2*, 51.1, 46.7, 44.3, 35.6, 33.5*, 31.1*, 30.0, 29.7, 29.5*, 24.8*, 24.5. HRMS (ESI) calcd for [M+]: C₉H₁₈N₄O₂: 214.2648. Found: 214.2647.

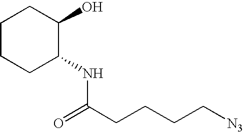

(2j) 5-azido-N-((trans)-2-hydroxycyclohexyl)pentanamide: Using General Procedure D using TBAF (7.90 mL, 79.0 mmol, 1M in tetrahydrofuran), trans 5-azido-N-((trans)-2-(tert-butyldimethylsilyloxy)cyclohexyl)pentanamide (1.40 g, 3.95 mmol) in tetrahydrofuran (32.0 mL). The reaction was purified using column chromatography (0-100% ethyl acetate in hexanes) to give a white solid (0.835 g, 88% yield). IR (cm⁻¹) 3288, 2934, 2860, 2095, 1640, 1550, 1451, 1264, 1070. ¹H NMR (500 MHz, CDCl₃) δ 5.79 (br s, 1H, —NH), 3.62 (m, 2H), 3.34-3.29 (m, 3H), 2.27 (t, J=7.3 Hz, 2H), 2.00 (dd, J=11.2, 49.3 Hz, 2H), 1.78-1.67 (m, 4H), 1.67-1.59 (m, 2H), 1.37-1.13 (m, 4H). ¹³C NMR (125 MHz, CDCl₃) δ 174.0, 74.0, 55.4, 50.8, 35.5, 34.1, 31.1, 28.0, 24.3, 23.8, 22.6. m.p. 59-60° C. HRMS (ESI) calcd for [M+Na]+: C₁₁H₂₀N₄O₂: 241.1659. Found: 241.1666.

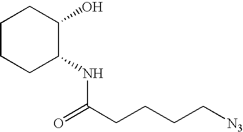

(2k) 5-azido-N-((cis)-2-hydroxycyclohexyl)pentanamide: Using General Procedure D using TBAF (7.90 mL, 79.0 mmol, 1 M in tetrahydrofuran), cis 5-azido-N-((trans)-2-(tert-butyldimethylsilyloxy)cyclohexyl)pentanamide (1.40 g, 3.95 mmol) in tetrahydrofuran (32.0 mL). The reaction was purified using column chromatography (0-100% ethyl acetate in hexanes). IR (cm⁻¹) 3318, 2932, 2860, 2091, 1628, 1531, 1447, 1247, 1132, 1062, 984, 888, 557. ¹H NMR (300 MHz, CDCl₃) δ 5.86 (br s, 1H, —NH), 3.93 (m, 2H), 3.30 (t, J=6.6 Hz, 2H), 2.22 (t, J=7.2 Hz, 2H), 2.06 (br s, 1H —OH), 1.63 (m, 12H). ¹³C NMR (75 MHz, CDCl₃) δ 172.1, 69.2, 51.2, 50.7, 36.1, 31.8, 28.3, 27.1, 23.7, 22.9, 19.7. m.p. 83-84° C. HRMS (ESI) calcd for [M+Na]+: C₁₁H₂₀N₄NaO₂. Found: 263.1478.

### General Procedure E

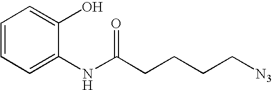

(2h) 4-azido-N-(2-hydroxyphenyl)butanamide: A round bottom flask was charged with 2-aminophenol (1.68 g, 15.4 mmol), tetrahydrofuran (100 mL) and triethylamine (2.34 mL, 16.8 mmol). At 0° C., a solution of 4-azidopentanoic acid (2.26 g, 13.4 mmol) in tetrahydrofuran (40 mL was added dropwise and the mixture was stirred for 1 hour at 0° C. then warmed to RT. The reaction was checked by TLC and determined to be complete. Then, the reaction quenched with sat. NH₄Cl and extracted in ethyl acetate (3×20 mL). Organic phase washed once with 0.5 M HCl and with brine, dried over MgSO₄, filtered and concentrated. The reaction was purified using column chromatography (30% ethyl acetate in hexanes) to provide the title compound as a yellow oil (2.76 g, 84% yield). IR (cm⁻¹) 3405, 3138, 2955, 2878, 2086, 1660, 1593, 1530, 1451, 1360, 1301, 1281, 1236, 1199, 1103. ¹H NMR (500 MHz, CDCl₃) δ 8.84 (br s, 1H), 8.10 (br s, 1H), 7.20 (d, J=7.9 Hz, 1H), 7.09 (t, J=7.6 Hz, 1H), 6.98 (d, J=8.1 Hz, 1H), 6.85 (t, J=7.6 Hz, 1H), 3.28 (t, J=5.5 Hz, 2H), 2.45 (t, J=6.6 Hz, 2H), 1.83-1.71 (m, 2H), 1.67-1.57 (m, 2H). ¹³C NMR (125 MHz, CDCl₃) δ 173.0, 148.0, 126.9, 125.5, 122.1, 120.5, 118.9, 51.0, 36.1, 28.1, 22.8. HRMS (ESI) calcd for [M+Na]+: C₁₁H₁₄N₄NaO₂: 257.1009. Found: 257.1009.

### Example 3

### Syntheses of Various Alkynyl Azide Compounds

### General Procedure B

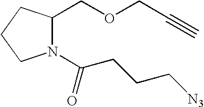

(3a) 4-azido-1-(2-((prop-2-ynyloxy)methyl)pyrrolidin-1-yl)butan-1-one: Sodium hydride (0.58 g, 14.4 mmol) was added to a round bottom flask equipped with a stir bar containing, 4-azido-1-(2-(hydroxymethyl)pyrrolidin-1-yl)butan-1-one (1.63 g, 7.22 mmol) and propargyl bromide (6.22 mL, 72.2 mmol) in tetrahydrofuran (68.0 mL) at 0° C., under N₂. The mixture was left at 0° C. for 30 min and warmed to RT slowly for another hour, after which the reaction was deemed complete by TLC and LCMS. The reaction was quenched up by slow addition of 10 mL of acetic acid (0.5 M) and extracted with ethyl acetate (10 mL×3), washed with brine, and dried with MgSO₄. The reaction was purified using column chromatography (20% ethyl acetate in hexanes). (1.70 g, 85% yield) IR (cm⁻¹) 2945, 2873, 2360, 2342, 2090, 1629, 1420, 1352, 1246, 1196, 1095, 1025, 954, 912, 669. ¹H NMR (2.6:1 rotamer ratio, asterisk denotes minor rotamer peaks, 300 MHz, CDCl₃) δ 4.28-4.20 (m, 1H), 4.14*(d, J=2.2 Hz, 1H), 4.11 (d, J=2.2 Hz, 1H), 3.65*(d, J=3.4 Hz, 1H), 3.62 (d, J=3.4 Hz, 1H), 3.56 (d, J=6.6 Hz, 1H), 3.53*(d, J=6.6 Hz, 1H), 3.43*(dt, J=2.9, 6.6 Hz, 2H), 3.36 (dt, J=6.6, 2.3 Hz, 2H), 2.57-2.39 (m, 2H), 2.34 (t, J=7.1 Hz, 2H), 2.05-1.82 (m, 8H). ¹³C NMR (2.6:1 rotamer ratio, asterisk denotes minor rotamer peaks 125 MHz, C₆D₆) δ 169.9, 80.6, 75.3*, 74.8, 71.5*, 70.4, 58.7, 56.8, 53.7*, 51.3, 47.1, 46.0*, 31.5, 30.9*, 29.0*, 27.9, 25.0*, 24.5, 24.5, 22.1*. HRMS (ESI) calcd for [M+Na]⁺: C₁₂H₁₈N₄NaO₂: 273.1322. Found: 273.1329.

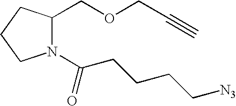

(3b) 4-azido-1-(2-(hydroxymethyl)pyrrolidin-1-yl)butan-1-one: This compound was prepared using General Procedure B using 4-azido-1-(2-(hydroxymethyl)pyrrolidin-1-yl)pentan-1-one (2.75 g, 12.2 mmol) propargyl bromide (14.5 g, 122 mmol) and sodium hydride (0.583 g, 24.3 mmol) to provide the title compound as a yellow oil (2.80 g, 87% yield). IR (cm⁻¹) 3232, 2944, 2873, 2090, 1628, 1421, 1353, 1245, 1196, 1168, 1094, 1025, 954, 912, 820, 730. ¹H NMR (2.4:1 rotamer ratio, asterisk denotes minor rotamer peaks, 500 MHz, CDCl₃) δ 4.29*(s, 2H), 4.15 (s, 2H), 3.72-3.57 (m, 2H), 3.56-3.37 (m, 2H), 3.31 (t, J=6.7 Hz, 2H), 2.48-2.37 (m, 1H), 2.32 (t, J=6.7 Hz, 2H), 2.15-1.85 (m, 4H), 1.77 (t, 2H), 1.70 (dd, J=7.4, 14.4 Hz, 2H), 1.56 (s, 1H). ¹³C NMR (2.9:1 rotamer ratio, asterisk denotes minor rotamer peaks, 125 MHz, CDCl₃) δ 171.8*, 171.4, 79.9, 79.3*, 75.1*, 74.4, 71.2*, 69.9, 58.6*, 58.5, 57.0*, 56.4, 51.4 (br, itself and rotamer), 47.4, 45.7*, 34.3, 33.6*, 28.9, 28.7*, 28.6*, 27.6, 24.3, 22.6*, 22.0, 21.9*. HRMS (ESI) calcd for [M+H]⁺: C₁₃H₂₁N₄O₂: 265.1659. Found: 265.1669.

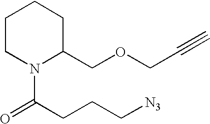

(3c) 4-azido-1-(2-((pr op-2-ynyloxy)methyl)piperidin-1-yl)butan-1-o n e: This compound was prepared using General Procedure B using 4-azido-1-(2-(hydroxymethyl)piperidin-1-yl)butan-1-one (0.93 g, 4.11 mmol), propargyl bromide (4.89 g, 41.1 mmol) and sodium hydride (0.197 g, 8.22 mmol) to provide the title compound as a yellow oil (0.70 g, 64% yield). IR (cm⁻¹) 3304, 2939, 2866, 2243, 2095, 1627, 1435, 1357, 1256 1177 1132, 1100, 1030, 908. ¹H NMR (1.1:1 rotamer ratio, asterisk denotes minor rotamer peaks, 500 MHz, CDCl₃) δ 4.88 (s, 1H), 4.50 (d, J=13.0 Hz, 1H), 4.22-3.98 (m, 2H), 3.76-3.47 (m, 2H), 3.31 (s, 2H), 3.07 (t, J=12.0 Hz, 1H)*, 3.57 (t, J=12.0 Hz, 1H), 2.50-2.25 (m, 2H), 1.97 (d, J=6.1 Hz, 1H), 1.91-1.78 (m, 1H), 1.72 (d, J=12.9 Hz, 1H), 1.67-1.40 (m, 4H), 1.39-1.24 (m, 1H), 1.20 (t, J=7.1 Hz, 1H). ¹³C NMR (1.1:1 rotamer ratio, asterisk denotes minor rotamer peaks, 125 MHz, CDCl₃) δ 171.7, 171.0*, 80.1*, 79.7, 75.2, 74.8*, 68.3, 67.8*, 58.8, 58.4*, 52.5*, 51.4, 51.3, 47.0*, 42.1, 37.3, 30.5*, 30.1, 26.8, 26.2*, 25.5*, 24.9, 20.0, 19.8*. HRMS (ESI) calcd for [M+Na]+: C₁₃H₂₀N₄NaO₂: 287.1479. Found: 287.1490.

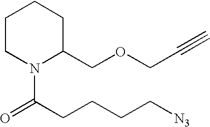

(3d) 5-azido-1-(2-((prop-2-ynyloxy)methyl)piperidin-1-yl)pentan-1-one: This compound was prepared using General Procedure B using 5-azido-1-(2-(hydroxymethyl)piperidin-1-yl)pentan-1-one (2.75 g, 11.4 mmol) propargyl bromide (13.6 g, 114 mmol) and sodium hydride (0.549 g, 22.9 mmol) to provide the title compound as a yellow oil (2.50 g, 78% yield). IR (cm¹) 3463, 3125, 2934, 2863, 2093, 1625, 1435, 1263, 1074, 1050. ¹H NMR (1.3:1 rotamer ratio, asterisk denotes minor rotamer peaks, 500 MHz, C₆D₆) δ 4.94 (s, 1H), 4.59 (d, J=11.7 Hz, 1H), 3.97-3.75 (m, J=18.7 Hz, 49.6, 4H), 3.41 (t, J=8.4 Hz, 2H)*, 3.22 (s, 1H), 2.88 (t, J=6.8 Hz, 2H), 2.69*(t, J=12.0 Hz, 1H), 2.52 (d, J=31.5 Hz, 2H), 2.34 (t, J=12.0 Hz, 1H), 2.24-1.97 (m, 2H), 1.92 (s, 1H), 1.67-1.01 (m, 2H), 1.31 (dd, J=8.5, 38.1 Hz, 6H). ¹³C NMR (1.3:1 rotamer ratio, asterisk denotes minor rotamer peaks, 125 MHz, C₆D₆) δ 170.9, 170.4*, 80.3*, 79.9, 75.4, 75.1*, 67.8, 67.5*, 58.3, 57.9*, 51.8*, 51.2, 46.5, 41.6, 36.6, 32.7*, 32.2, 28.5, 26.4, 25.9*, 25.4, 25.2*, 22.5*, 19.8, 19.45*. HRMS (ESI) calcd for [M+H]⁺: C₁₄H₂₃N₄O₂: 279.1816. Found: 279.1818.

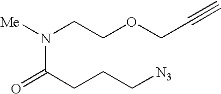

(3e) 4-azido-N-methyl-N-(2-(prop-2-ynyloxy)ethyl)butanamide: This compound was prepared using General Procedure B using 4-azido-N-(2-hydroxyethyl)-N-methylbutanamide (1.00 g, 5.37 mmol), 3-bromoprop-1-yne (4.63 mL, 53.7 mmol), NaH (0.258 g, 10.7 mmol) in tetrahydrofuran (45 mL) at to provide the title compound as a yellow oil (1.00 g, 83% yield). IR (cm⁻¹) 3296, 2939, 2365, 2100, 1630, 1465, 1408, 1256, 1096, 713. ¹H NMR (300 MHz, CDCl₃) δ 4.10 (dd, J=2.4, 3.8 Hz, 3H), 3.61 (q, J=5.7 Hz, 3H), 3.53 (t, J=4.9 Hz, 2H), 3.47 (t, J=5.3 Hz, 1H), 3.33 (dd, J=5.7, 12.2 Hz, 4H), 3.03 (s, 3H), 2.91 (s, 2H), 2.50-2.30 (m, 5H), 1.88 (p, J=6.8 Hz, 4H). ¹³C NMR (1.2:1 rotamer ratio, asterisk denotes minor rotamer peaks, 75 MHz, CDCl₃) δ 172.2*, 171.8, 79.6, 79.2*, 74.9*, 74.5, 68.4, 67.2*, 58.2, 51.0*, 50.9, 49.4*, 47.7, 36.8, 33.7*, 30.0, 29.5*, 24.5*, 24.2. HRMS (ESI) calcd for [M+Na]⁺: C₁₀H₁₆N₄NaO₂: 247.1166. Found: 247.1172.

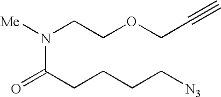

(3f) 4-azido-N-methyl-N-(2-(prop-2-ynyloxy)ethyl)pentanamide: This compound was prepared using General Procedure B using (S)-4-azido-N-(2-hydroxyethyl)-N-methylpentanamide (3.00 g, 15.0 mmol) propargyl bromide (17.8 g, 12.9 mmol) and sodium hydride (0.719 g, 30.0 mmol) to provide the title compound as a yellow oil (3.00 g, 84% yield). IR (cm⁻¹) 3302, 2938, 2244, 2093, 1633, 1466, 1403, 1352, 1267, 1103, 1031, 908, 822. ¹H NMR (1.1:1 rotamer ratio, asterisk denotes minor rotamer peaks 500 MHz, C₆D₆) δ 3.91 (d, J=2.4 Hz, 2H), 3.84*(d, J=2.3 Hz, 2H), 3.54*(t, J=5.3 Hz, 2H), 3.45 (t, J=5.3 Hz, 2H), 3.21*(t, J=5.5 Hz, 2H), 2.99 (t, J=5.5 Hz, 2H), 2.88 (dt, J=7.0, 11.0 Hz, 2H), 2.81*(s, 3H), 2.57 (s, 3H), 2.41*(t, J=2.3 Hz, 1H), 2.36 (t, J=2.3 Hz, 2H), 2.10*(t, J=7.1 Hz, 2H), 1.87 (t, J=7.2, 2H), 1.75 (s, 1H), 1.70-1.53 (m, 1H), 1.43-1.33 (m, 1H). ¹³C NMR (1.4:1 rotamer ratio, asterisk denotes minor rotamer peaks, 125 MHz, C₆D₆) δ 172.2*, 172.0, 80.7, 80.3*, 75.9*, 75.5, 69.3, 67.9*, 58.9*, 58.7, 51.8, 49.5*, 48.2, 36.9, 33.8*, 33.2, 32.6*, 29.3*, 29.2, 23.1*, 22.8. HRMS (ESI) calcd for [M+Na]⁺: C₁₁H₁₈N₄NaO₂: 261.1322. Found: 261.1323.

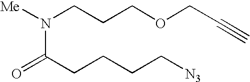

(3g) 5-azido-N-methyl-N-(3-(prop-2-ynyloxy)propyl)pentanamide: This compound was prepared using General Procedure B using 5-azido-N-(3-(tert-butyldimethylsilyloxy)propyl)-N-methylpentanamide (2.0 g, 9.99 mmol), propargyl bromide (8.61 g, 10.0 mmol) and sodium hydride (0.479 g, 20.0 mmol) in tetrahydrofuran 100 mL to provide the title compound as a yellow oil (2.0 g, 84% yield). IR (cm⁻¹) 3293, 3090, 3035, 2932, 2094, 1640, 1478, 1402, 1350, 1258, 1199, 1102, 1035, 922, 733, 676. ¹H NMR (1.1:1 rotamer ratio, asterisk denotes minor rotamer peaks, 500 MHz, CDCl₃) δ 3.96 (d, J=2.5 Hz, 2H), 3.92 (d, J=2.5 Hz, 2H)*, 3.38 (t, J=6.0 Hz, 2H), 3.36 (t, J=7.0 Hz, 2H)*, 3.21 (t, J=6.0 Hz, 2H), 3.11 (t, J=6.5 Hz, 2H)*, 3.08 (t, J=7.0 Hz, 2H)*, 3.01 (t, J=7.0 Hz, 2H), 2.74 (s, 3H)*, 2.52 (t, J=2.5 Hz, 1H)*, 2.48 (t, J=2.5 Hz, 1H), 2.43 (s, 3H), 2.17 (t, J=7.0 Hz, 2H)*, 1.96 (t, J=7.0 Hz, 2H), 1.85 (p, J=7.0 Hz, 2H)*, 1.78 (p, J=7.0 Hz, 2H), 1.71 (p, J=7.0 Hz, 2H)*, 1.47 (p, J=6.5 Hz, 2H). ¹³C NMR (1.1:1 rotamer ratio, asterisk denotes minor rotamer peaks, 125 MHz, CDCl₃) δ 170.7*, 170.6, 80.3, 79.9*, 75.1*, 74.8, 67.6, 66.3*, 58.0, 58.0*, 51.2, 51.0*, 45.9*, 45.2, 34.9, 32.6*, 29.7, 29.1*, 28.2*, 27.8, 24.6*, 24.4. HRMS (ESI) calcd for [M+H]⁺: C₁₂H₂₁N₄O₂: 239.1503.

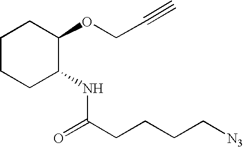

(3j) 5-azido-N-((trans)-2-hydroxycyclohexyl)pentanamide. Using General Procedure B using NaH (0.276 g, 6.91 mmol), propargyl bromide (5.14 g, 34.5 mmol) and 5-azido-N-((trans)-2-hydroxycyclohexyl)pentanamide (0.830 g, 3.45 mmol) in DMF (30 mL). The reaction was purified using column chromatography (50% ethyl acetate in hexanes) to yield the title compound as a yellow oil (0.770 g, 80% yield). IR (cm⁻¹): 3293 (br), 2933, 2859, 2092, 1636, 1546, 1244, 1086. ¹H NMR (500 MHz, CDCl₃) δ 5.65 (s, 1H), 4.25 (dd, J=2.4, 16.2 Hz, 1H), 4.12 (dd, J=2.4, 16.2 Hz, 1H), 3.72-3.62 (m, 1H), 3.37-3.25 (m, 3H), 2.45 (t, J=2.3 Hz, 1H), 2.20 (t, J=7.2 Hz, 3H), 2.06 (m, 1H), 1.80-1.68 (m, 3H), 1.64 (m, 3H), 1.27 (m, 3H), 1.19-1.08 (m, 1H). ¹³C NMR (125 MHz, CDCl₃) δ 172.2, 80.7, 78.7, 74.1, 55.5, 52.9, 51.1, 36.2, 31.5, 29.7, 28.3, 24.0, 23.9, 22.8. HRMS (ESI) calcd for [M+Na]⁺: C₁₄H₂₂N₄NaO₂: 301.1635. Found: 301.1632.

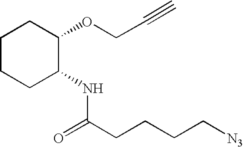

(3k) 5-azido-N-((cis)-2-hydroxycyclohexyl)pentanamide: Using General Procedure B using NaH (0.64 g, 2.66 mmol), propargyl bromide (1.54 g, 13.3 mmol) and 5-azido-N-((cis)-2-hydroxycyclohexyl)pentanamide (0.32 g, 1.33 mmol) in DMF (13 mL). The reaction was purified using column chromatography (50% ethyl acetate in hexanes) to yield the title compound as a yellow oil (0.33 g, 81% yield). IR (cm⁻¹) 3300, 2935, 2861, 2095, 1641, 1514, 1448, 1352, 1253, 1130, 1080, 1056, 669. ¹H NMR (500 MHz, CDCl₃) δ 5.91 (br s, 1H, —NH), 4.25 (d, J=16.0 Hz, 1H), 4.07 (d, J=16.0 Hz, 1H), 3.93 (m, 1H), 3.74 (s, 1H), 3.29 (t, J=6.7 Hz, 2H), 2.42 (s, 1H), 2.20 (t, J=7.3 Hz, 2H), 1.94 (d, J=12.5 Hz, 1H), 1.72 (m, 2H), 1.64 (m, 4H), 1.49 (s, 1H), 1.39 (m, 4H). ¹³C NMR (125 MHz, CDCl₃) δ 171.2, 80.0, 75.1, 74.1, 55.5, 51.0, 49.4, 35.9, 28.2, 27.3, 27.2, 24.0, 22.7, 19.3. HRMS (ESI) calcd for [M+Na]⁺: C₁₄H₂₂N₄NaO₂: 301.1635. Found: 301.1632. Found: 239.1509.

### General Procedure F

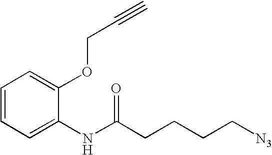

(3h) 4-azido-N-(2-(prop-2-ynyloxy)phenyl)butanamide: Potassium carbonate (1.52 g, 11.0 mmol) was added to a round bottom flask with a stir bar containing 4-azido-N-(2-hydroxyphenyl)butanamide (2.58 g, 11.0 mmol) in DMF (22.0 mL) at 0° C., then 3-bromoprop-1-yne (1.24 mL, 13.2 mmol) was added slowly to the mixture. The reactions warmed to RT and stirred overnight, under N₂. The reaction was determined to be complete using TLC and LCMS. Then the reaction mixture was concentrated and quenched with water. The oily residue was extracted with ethyl acetate (3×20 mL). The reaction was purified using column chromatography (0-30% methanol in dichloromethane) to provide the title compound as an oil (2.56 g, 85% yield). IR (cm⁻¹) 3411, 3289, 2935, 2869, 2091, 1675, 1599, 1519, 1481, 1449, 1290, 1246, 1199, 1159, 1115, 1047, 1021, 926, 746, 629, 553, 450, 405. ¹H NMR (300 MHz, CDCl₃) δ 8.37 (d, J=7.6 Hz, 1H), 7.75 (br s, 1H, —NH), 7.09-6.92 (m, 3H), 4.75 (d, J=2.4 Hz, 2H), 3.32 (t, J=6.7 Hz, 2H), 2.57 (t, J=2.4 Hz, 1H), 2.43 (t, J=7.2 Hz, 2H), 1.75-1.86 (m, 2H), 1.73-1.61 (m, 2H). ¹³C NMR (125 MHz, CDCl₃) δ 170.4, 145.7, 128.0, 123.5, 122.1, 120.1, 111.7, 77.9, 76.2, 56.6, 51.1, 37.1, 28.3, 22.6. HRMS (ESI) calcd for [M+Na]⁺: C₁₄H₁₆N₄NaO₂: 295.1165. Found: 295.1163.

### General Procedure G

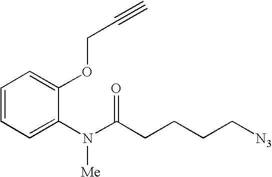

(3i) 4-azido-N-methyl-N-(2-(prop-2-ynyloxy)phenyl)butanamide: A round bottom flask with stir bar was charged with 4-azido-N-(2-(prop-2-ynyloxy)phenyl)butanamide (1.400 g, 5.14 mmol) in tetrahydrofuran (51 mL) under a blanket of N₂. The reaction mixture was cooled to 0° C. and NaHMDS (5.66 mL, 5.66 mmol, 1 M in tetrahydrofuran) was added slowly. Then, methyl iodide (0.483 mL, 7.71 mmol) was added dropwise to the mixture. The reaction was warmed to RT and allowed to stir for 1 h. Then, the reaction was determined to be complete using TLC and LCMS. The reaction mixture quenched with water and aq. NH₄Cl and the tetrahydrofuran was removed in vacuo. The oily residue was extracted with ethyl acetate (3×20 mL) and dried over MgSO₄. The reaction was purified using column chromatography (30% ethyl acetate in hexanes) to give the title compound as an oil (1.17 g, 80% yield). IR (cm⁻¹) 3303, 3218, 2934, 2873, 2093, 1646, 1596, 1497, 1455, 1386, 1281, 1220, 1021, 904, 727, 648. ¹H NMR (300 MHz, CDCl₃) δ 7.38-7.29 (m, 1H), 7.18-7.07 (m, 2H), 7.03 (td, J=1.2, 7.6, 1H), 4.74 (dd, J=1.0, 2.3 Hz, 2H), 3.22-3.12 (m, 5H), 2.49 (t, J=2.4 Hz, 1H), 2.03 (t, J=7.1 Hz, 2H), 1.70-1.57 (m, 2H), 1.56-1.45 (m, 2H). ¹³C NMR (75 MHz, CDCl₃) δ 173.0, 152.8, 132.8, 129.3, 129.1, 122.0, 113.4, 77.8, 76.0, 55.6, 51.1, 36.0, 32.7, 28.3, 22.2. HRMS (ESI) calcd for [M+Na]⁺: C₁₅H₁₈N₄NaO₂: 309.1322. Found: 309.1324.

### Example 4

### Synthesis of Various Macrocycles via Ruthenium-Catalyzed Intramolecular Huisgen Cycloaddition

### General Procedure H

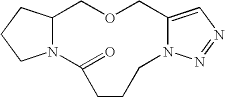

(4a) A flame dried round bottom flask containing a stir bar was charged with 4-azido-1-(2-((prop-2-ynyloxy)methyl)pyrrolidin-1-yl)butan-1-one (370 mg, 1.48 mmol) in toluene (730 mL) the reaction mixture was sparged with N₂ for 30 min at 80° C. Then, [Cp*RuCl]₄ (0.80 g, 74 μmol) was added. The reaction was monitored by LCMS. The reaction was deemed complete by TLC and LCMS in approximately 10 min. The solvent was removed in vacuo. The product was purified using column chromatography (5% methanol in dichloromethane) to yield the title compound as a white solid (210 mg, 58% yield). IR (cm⁻¹) 2958, 2360, 2341, 1619, 1440, 1356, 1328, 1232, 1179, 1108, 1021, 669. ¹H NMR (500 MHz, CDCl₃) δ 7.58 (s, 1H), 4.68 (d, 1H, J=11.5 Hz), 4.58 (dd, 1H, J=5, 13.5 Hz), 4.36 (d, 1H, J=11.0 Hz), 4.17 (t, 1H, J=8.5 Hz), 4.03 (t, 1H, J=12.5 Hz), 3.71 (t, 1H, J=9.5 Hz), 3.52 (1H, m), 3.34 (t, 1H, J=10.0 Hz), 3.13 (t, 1H, J=10.0 Hz), 2.81 (m, 1H), 1.91 (m, 3H), 1.84 (d, 2H, J=8.5 Hz) and 1.68 (m, 1H). ¹³C NMR (125 MHz, CDCl₃) δ 172.0, 133.5, 132.9, 73.5, 60.9, 57.0, 47.2, 45.8, 30.1, 28.7, 27.2, 22.4 ppm. HRMS (ESI) calcd for C₁₂H₁₈N₄NaO₂ [M+Na]⁺: 273.1322. Found: 273.1312.

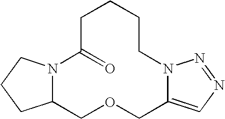

(4b) This compound was prepared using General Procedure H using 4-azido-1-(2-((prop-2-ynyloxy)methyl)pyrrolidin-1-yl)pent-1-one (180 mg, 0.681 mmol), and [Cp*RuCl]₄ (0.37 g, 34 μmol) in toluene (340 mL) to provide the title compound as a white solid (121 mg, 67% yield). IR (cm⁻¹) 2950, 2874, 2360, 1614, 1447, 1419, 1351, 1232, 1188, 1097, 977, 913, 834, 723. ¹H NMR (1.1:1 rotamer ratio, asterisk denotes minor rotamer peaks, 300 MHz, CDCl₃) δ 7.54*(s, 1H), 7.48 (s, 1H), 4.66 (d, J=11.3 Hz, 2H), 4.52*(d, J=12.1 Hz, 2H), 4.47-4.05 (m, 4H), 3.64 (m, 1H), 3.56-3.33 (m, 2H), 3.32-3.22 (m, 1H), 2.95-2.70 (m, 1H), 2.58-2.41*(m, 1H), 2.33-2.11*(m, 3H), 2.11-1.96 (m, 3H), 1.95-1.55 (m, 4H), 1.51-1.30 (m, 1H). ¹³C NMR (1.1:1 rotamer ratio, asterisk denotes minor rotamer peaks, 75 MHz, CDCl₃) δ 172.7, 172.0*, 133.7*, 133.2, 132.9*, 132.3, 73.5, 69.5*, 61.8, 60.9*, 56.9, 56.5*, 48.8*, 48.0, 46.4*, 45.1, 34.3*, 31.8, 29.3, 28.7*, 28.5, 27.4*, 24.5*, 22.3, 21.23*, 21.0. HRMS (ESI) calcd for [M+H]⁺: C₁₃H₂₁N₄O₂: 265.1659. Found: 265.1661.

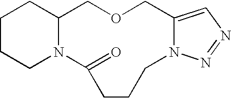

(4c) This compound was prepared using General Procedure H using 4-azido-1-(2-((prop-2-ynyloxy)methyl)piperidin-1-yl)butan-1-one (210 mg, 0.794 mmol), and [Cp*RuCl]₄ (0.43 g, 40 μmol) in toluene (378 mL) to provide the title compound as a yellow oil (138 mg, 66% yield). IR (cm⁻¹) 2935, 2868, 2097, 1689, 1628, 1445, 1392, 1366, 1247, 1175, 1151, 1093. ¹H NMR (2:1 rotamer ratio, asterisk denotes minor rotamer peaks, 500 MHz, CDCl₃) δ 7.61 (s, 1H), 7.59*(d, J=6.1 Hz, 1H), 4.93*(d, J=13.6 Hz, 2H), 4.41 (d, J=13.5 Hz, 2H), 4.38-4.22 (m, 2H), 4.21-4.12*(m, 1H), 4.03 (s, OH), 3.80*(t, J=8.7 Hz, 2H), 3.66 (t, J=10.0 Hz, 2H), 3.50-3.29*(m, 2H), 3.23 (dd, J=3.1, 9.1 Hz, 2H), 2.64-2.52*(m, 2H), 2.40-2.26 (m, 2H), 2.24-2.09 (m, 2H), 2.06-1.91*(m, 2H), 1.70-1.46 (m, 7H), 1.45-1.17*(m, 7H). ¹³C NMR (125 MHz, CDCl₃) δ 171.2, 135.4, 131.8, 66.7, 59.8, 51.6, 48.3, 36.0, 29.9, 26.6, 25.8, 25.2, 20.1. HRMS (ESI) calcd for [M+H]⁺: C₁₃H₂₁N₄O₂: 265.1659. Found: 265.1662.

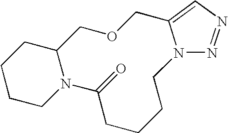

(4d) This compound was prepared using General Procedure H using 4-azido-1-(2-((prop-2-ynyloxy)methyl)piperidin-1-yl)pent-1-one (210 mg, 0.754 mmol), and [Cp*RuCl]₄ (0.41 g, 38 μmol) in toluene (359 mL) to provide the title compound as a yellow oil (121 mg, 58% yield). IR (cm⁻¹) 2932, 2859, 2093, 1725, 1681, 1432, 1357, 1259, 1177, 1132, 1098, 1029, 911. ¹H NMR (1.1:1 rotamer ratio, asterisk denotes minor rotamer peaks, 500 MHz, CDCl₃) δ 7.58 (s, 1H), 7.41 (s, 1H)*, 5.08 (s, 1H), 4.71-4.46 (m, 3H)*, 4.43-4.19 (m, 3H), 3.94 (t, J=9.8 Hz, 1H)*, 3.73 (t, J=9.8 Hz, 1H), 3.49-3.23 (m, 2H), 2.79 (s, 1H)*, 2.70-2.42 (m, 2H), 2.23-2.08 (m, 2H)*, 2.02 (s, 1H)*, 1.86 (s, 2H), 1.74-1.46 (m, 8H), 1.45-1.20 (m, 4H)*, 1.13 (t, J=7.0 Hz, 1H). ¹³C NMR (1.1:1 rotamer ratio, asterisk denotes minor rotamer peaks, 125 MHz, CDCl₃) δ 173.3, 172.9*, 135.4, 132.6, 69.0*, 67.1, 62.6*, 59.5, 53.1*, 48.6, 47.6*, 46.7, 41.9, 36.8*, 34.0, 30.1*, 29.5*, 27.1, 26.2*, 25.43*, 25.3, 21.5, 20.2*, 19.9, 19.9. HRMS (ESI) calcd for [M+H]⁺: C₁₄H₂₃N₄O₂: 279.1816. Found: 279.1828.

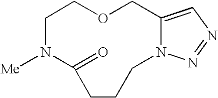

(4e) This compound was prepared using General Procedure H using 4-azido-N-methyl-N-(2-(prop-2-ynyloxy)ethyl)butanamide (213 mg, 0.95 mmol), and [Cp*RuCl]₄ (0.52 g, 47 μmol) in toluene (475 mL) to provide the title compound as a white solid (100 mg, 47% yield). IR (cm⁻¹) 3471, 2932, 2873, 1632, 1439, 1400, 1355, 1266, 1216. ¹H NMR (500 MHz, DMSO at 150° C.) δ 7.58 (s, 1H), 4.61 (s, 2H), 4.30 (t, J=5.0 Hz, 2H), 3.73 (t, J=4.5 Hz, 2H), 3.42 (t, J=4.5 Hz, 2H), 2.77 (s, 3H), 2.45-2.39 (m, 2H), 2.38-2.31 (m, 2H). ¹³C NMR (125 MHz, CDCl₃) δ 173.1, 134.0, 133.2, 67.7, 61.4, 50.8, 47.2, 34.1, 28.0, 26.9. m.p. 180-181° C. HRMS (ESI) calcd for [M+Na]⁺: C₁₀H₁₆N₄NaO₂: 247.1166. Found: 247.1164.

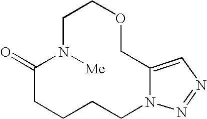

(4f) This compound was prepared using General Procedure H using 4-azido-N-methyl-N-(2-(prop-2-ynyloxy)ethyl)pentanamide (149 mg, 0.625 mmol), and [Cp*RuCl]₄ (0.34 g, 31 μmol) in toluene (313 mL) to provide the title compound as a yellow oil (80 mg, 54% yield). IR (cm⁻¹) 2932, 2871, 2096, 1627, 1456, 1403, 1355, 1263, 1103. ¹H NMR (2.5:1 rotamer ratio, asterisk denotes minor rotamer peaks, 300 MHz, CDCl₃) δ 7.54 (s, 1H)*, 7.42 (s, 1H), 4.84-4.64 (m, 1H) 4.56 (s, 2H), 4.29 (t, J=7.1 Hz, 2H), 3.72-3.50 (m, 4H), 2.90 (s, 3H), 2.73 (s, 3H)*, 2.39 (t, J=6.4 Hz, 2H), 2.22-2.01 (m, 1H), 1.93-1.80 (m, 1H), 1.75-1.62 (m, 1H). ¹³C NMR (75 MHz, CDCl₃) δ 173.9, 132.5, 132.3, 67.7, 62.1, 50.5, 47.0, 33.2, 30.4, 28.9, 21.3. HRMS (ESI) calcd for [M+Na]⁺: C₁₁H₁₈N₄NaO₂: 261.1322. Found: 261.1330.

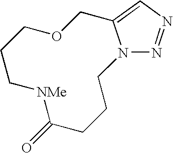

(4g) This compound was prepared using General Procedure H using 5-azido-N-methyl-N-(3-(prop-2-ynyloxy)propyl)pentanamide (90 mg, 0.378 mmol), and [Cp*RuCl]₄ (21 mg, 19 μmol) in toluene (189 mL) to provide the title compound as a yellow oil (55 mg, 61% yield). IR (cm⁻¹) 2934, 2242, 1631, 1478, 1456, 1360, 1291, 1295, 1178, 1095, 1040, 969, 907, 839, 723, 645. ¹H NMR (6.7:1 rotamer ratio, asterisk denotes minor rotamer peaks, 500 MHz, CDCl₃) δ 7.65 (s, 1H), 7.44*(s, 1H), 4.68 (d, J=13.1 Hz, 1H), 4.63*(t, J=6.5 Hz, 1H), 4.59-4.46 (m, 2H), 4.42 (d, J=13.1 Hz, 1H), 4.38 (dt, J=4.0, 13.3 Hz, 1H), 3.44*(t, J=5.3 Hz, 2H), 3.37-3.31 (m, 1H), 3.12-3.06 (m, 1H), 3.06-2.96 (m, 1H), 2.84*(s, 3H), 2.57 (s, 3H), 2.37 (d, J=13.6 Hz, 1H), 2.23-2.10 (m, 3H), 2.06-1.98 (m, 1H), 1.81-1.69 (m, 1H), 1.65-1.54 (m, 1H). ¹³C NMR (50:1 rotamer ratio, asterisk denotes minor rotamer peaks, 125 MHz, CDCl₃) δ 171.9, 145.0, 125.6, 66.2*, 63.8*, 62.8, 61.9, 49.7, 47.8*, 47.0*, 41.3, 32.6, 28.8, 28.1*, 27.1*, 26.0, 24.8*, 22.3. HRMS (ESI) calcd for [M+H]⁺: C₁₁H₁₈N₄O₂: 239.1503. Found: 239.1508.

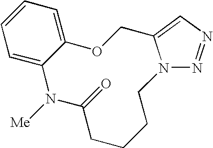

(4h) This compound was prepared using General Procedure H using 4-azido-N-methyl-N-(2-(prop-2-ynyloxy)phenyl)butanamide (200 mg, 0.698 mmol), and [Cp*RuCl]₄ (0.38 g, 35 μmol) in toluene (349 mL) to provide the title compound as a yellow oil (96 mg, 48% yield). IR (cm⁻¹) 3226, 3026, 2943, 1648, 1540, 1495, 1455, 1302, 1262, 1110, 1038, 1008, 864, 755, 737. ¹H NMR (500 MHz, CDCl₃) δ 7.62 (s, 1H), 7.33 (td, J=1.5, 8.0 Hz, 1H), 7.14 (dd, J=1.4, 7.7, 1H), 7.06 (d, J=8.2, 1H), 7.03 (t, J=7.6, 1H), 5.22 (d, J=11.9, 1H), 5.01 (d, J=11.9, 1H), 4.32 (ddd, J=6.7, 10.1, 13.5, 1H), 3.94-3.86 (m, 1H), 3.07 (s, 3H), 2.23-2.14 (m, 1H), 2.11-2.02 (m, 1H), 1.99-1.88 (m, 2H), 1.69-1.58 (m, 1H), 1.41-1.31 (m, 1H). ¹³C NMR (126 MHz, CDCl3) δ 172.7, 152.7, 133.7, 132.5, 130.5, 129.6, 129.6, 122.6, 112.5, 58.8, 46.6, 36.8, 30.4, 28.7, 21.0. HRMS (ESI) calcd for [M+]⁺: C₁₅H₁₈N₄O₂: 286.3290. Found: 286.3289.

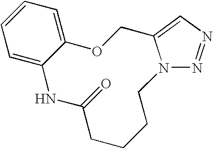

(4i) This compound was prepared using General Procedure H using 4-azido-N-(2-(prop-2-ynyloxy)phenyl)butanamide (200 mg, 0.698 mmol), and [Cp*RuCl]₄ (38 mg, 35 μmol) in toluene (349 mL) to provide the title compound as a white solid (128 mg, 64% yield). IR (cm⁻¹) 3226, 3026, 2944, 1648, 1540, 1495, 1455, 1262, 1110, 1008, 755, 737. ¹H NMR (1: 0.3 rotamer ratio, asterisk denotes minor rotamer peaks, 500 MHz, CDCl3) δ 7.74*(br s, 1H), 7.65 (s, 1H), 7.43-7.29 (m, 2H), 7.20-7.03 (m, 2H, NH), 6.80*(br s, 1H), 5.11 (s, 2H), 4.59-4.31 (m, 2H), 4.31-4.20*(m, 2H), 2.44 (br s, 2H), 2.23 (br s, 2H), 2.16*(br s, 2H), 2.04*(br s, 2H), 1.86 (br s, 2H), 1.79 (br s, 1H), 1.71*(br s, 2H). ¹³C NMR (125 MHz, CD₃OD) δ 176.7, 155.1, 134.4, 134.2, 130.6, 130.2, 128.6, 123.9, 116.5, 61.4, 49.9, 37.1, 30.4, 22.1. m.p. 238-241° C. HRMS (ESI) calcd for [M+H]⁺: C₄H₇N₄O₂: 273.1346. Found: 273.1345.

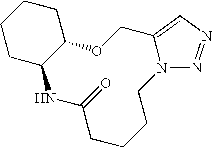

(4j) This compound was prepared using General Procedure H using 5-azido-N-((trans)-2-hydroxycyclohexyl)pentanamide (190 mg, 683 mmol), and [Cp*RuCl]₄ (37 mg, 34 μmol) in toluene (340 mL) to provide the title compound as a white solid (135 mg, 71% yield). IR (cm⁻¹): 3274 (br), 2935, 2859, 1639, 1554, 1085, 730. ¹H NMR (500 MHz, CDCl₃) δ 7.60 (s, 1H), 6.31 (s, br, 1H), 4.73 (d, J=12.6 Hz, 1H), 4.55 (d, J=12.6 Hz, 1H), 4.54 (m, 1H) 4.45 (m, 1H), 3.84 (m, 1H), 3.19 (m, 1H), 2.31 (m, 2H), 2.21 (m, 1H), 2.08 (m, 2H), 1.94 (m, 2H), 1.82 (d, J=10.5 Hz, 1H), 1.72 (d, J=12.0 Hz, 1H), 1.52 (m, 1H), 1.28 (m, 4H). ¹³C NMR (125 MHz, CDCl₃) δ 173.0, 133.9, 133.0, 80.4, 57.6, 52.7, 48.4, 35.7, 32.2, 29.9, 27.8, 24.6, 24.2, 21.9. m.p. decomposed at 270° C. HRMS (ESI) calcd for [M+]⁺: C₁₄H₂₂N₄O₂: 278.3501. Found: 278.3504.

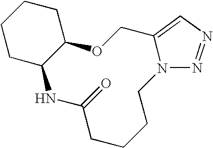

(4k) This compound was prepared using General Procedure H using 5-azido-N-((cis)-2-hydroxycyclohexyl)pentanamide (200 mg, 719 mmol), and [Cp*RuCl]₄ (39 mg, 36 μmol) in toluene (360 mL) to provide the title compound as a yellow oil (14 mg, 70% yield). IR (cm⁻¹): 3323 (br), 2935, 2858, 1636, 1542, 1081, 908, 725. ¹H NMR (500 MHz, CDCl₃) δ 7.59 (s, 1H), 5.68 (br s, 1H, —NH), 4.72 (d, J=11.9 Hz, 1H), 4.41 (m, 1H), 4.26 (d, J=11.9 Hz, 1H), 4.24 (m, 1H), 4.01 (m, 2H), 2.38 (m, 1H), 2.26 (m, 1H), 2.13 (m, 1H), 1.88 (m, 3H), 1.76 (m, 1H), 1.59 (m, 3H), 1.54 (m, 2H), 1.44 (m, 2H). ¹³C NMR (125 MHz, CDCl₃) δ 172.7, 133.5, 133.0, 75.5, 58.4, 49.7, 48.1, 36.0, 27.4, 27.0, 26.6, 23.4, 21.5, 20.0. m.p. 215-216° C. HRMS (ESI) calcd for [M+]⁺: C₁₄H₂₂N₄O₂: 278.3501. Found: 278.3499.

### Example 5

### Synthesis of Various Macrocycles via Copper-Catalyzed Intramolecular Huisgen Cycloaddition

### General Procedure I

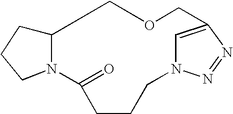

(5a) A flame dried round bottom flask containing a stir bar was charged with 4-azido-1-(2-((prop-2-ynyloxy)methyl)pyrrolidin-1-yl)butan-1-one (236 mg, 0.659 mmol) in toluene (66 mL) the reaction mixture was sparged with N₂ for 30 min. Then, at 60° C. Amberlyst-21 CuPF₆ (3.0 g, 0.659 mol, 0.21 mmol/g) was added. The reaction was monitored by LCMS. The reaction was deemed complete by TLC and LCMS in approximately 16 h. The beads were filtered off and solvent was removed in vacuo. The product was purified using column chromatography (5% methanol in dichloromethane) to yield the title compound as a white solid (85 mg, 52% yield). IR (cm⁻¹) 3053, 2972, 2928, 2872, 1622, 1433, 1353, 1322, 1265, 1232, 1160, 1127, 1101, 1091, 1054, 1020. ¹H NMR (500 MHz, CDCl₃) δ 7.49 (s, 1H), 4.78 (d, J=12.7 Hz, 1H), 4.50 (br m, 2H), 4.18 (br s, 1H), 3.78 (d, J=12.0 Hz, 1H), 3.71 (s, 1H), 3.63 (br s, 1H), 3.02 (br m, 2H), 2.85 (s, 1H), 2.08 (s, 2H), 2.01 (ddd, J=7.5, 15.1, 19.3 Hz, 2H), 1.90 (dt, J=5.8 Hz, 17.1, 1H), 1.72 (br s, 1H), 1.60 (br s, 1H). ¹³C NMR (10:1 rotamer ratio, asterisk denotes minor rotamer peaks, 125 MHz, CDCl₃) δ 170.71 (br), 169.05*, 145.92 (br), 144.42*, 123.94, 69.15 (br), 68.15*, 63.94, 58.27, 56.09*, 50.67*, 49.62 (br), 47.90, 46.42*, 31.08*, 29.90 (br), 28.57*, 28.14*, 26.81, 24.71, 24.43, 22.31*. ¹H NMR (500 MHz, DMSO at 120° C.) δ 7.82 (s, 1H), 4.67 (d, J=12.9 Hz, 1H), 4.61-4.52 (m, 1H), 4.32 (dt, J=4.0, 13.1 Hz, 1H), 4.21 (d, J=12.9 Hz, 1H), 3.68 (d, J=11.4 Hz, 1H), 3.63 (s, 1H), 3.42 (d, J=11.4 Hz, 1H), 3.26 (t, J=6.0 Hz, 2H), 2.60-2.50 (m, 1H), 2.33-2.24 (m, 1H), 2.16-2.07 (m, 1H), 2.02-1.82 (m, 3H), 1.82-1.73 (m, 1H), 1.69-1.58 (m, 1H). ¹³C NMR (125 MHz, DMSO at 120° C.) δ 168.1, 144.3, 124.0, 66.0, 62.5, 56.6, 48.9, 46.5, 30.1, 25.8, 23.7, 23.4. m.p. 160-161° C. HRMS (ESI) calcd for [M+H]⁺: C₁₂H₁₈N₄O₂: 251.1503. Found: 251.1502.

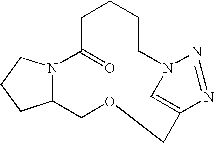

(5b) This compound was prepared using General Procedure I using 4-azido-1-(2-((prop-2-ynyloxy)methyl)pyrrolidin-1-yl)pentan-1-one (191 mg, 0.72 mmol) Amberlyst-21 CuPF₆ (3.40 g, 0.72 mol, 0.21 mmol/g) in toluene (72 mL) to provide the title compound as a yellow oil (107 mg, 56% yield). IR (cm⁻¹) 3126, 3053, 2948, 2930, 2917, 2867, 1629, 1440, 1425, 1265, 1102, 1049, 1030, 1011. ¹H NMR (500 MHz, CDCl₃) δ 7.53 (s, 1H), 4.78 (d, J=13.2 Hz, 1H), 4.57 (t, J=12.9 Hz, 1H), 4.32 (dd, J=6.1, 14.0 Hz, 1H), 4.11 (d, J=13.2 Hz, 1H), 3.85 (d, J=8.8 Hz, 1H), 3.73 (d, J=11.5 Hz, 1H), 3.66 (d, J=11.5 Hz, 1H), 2.97 (td, J=4.1, 9.2 Hz, 1H), 2.88 (dd, J=7.9, 17.4 Hz, 1H), 2.54 (dt, J=11.5, 13.9 Hz, 1H), 2.11 (dd, J=9.1, 20.6 Hz, 1H), 2.06-1.94 (m, 2H), 1.93-1.90 (m, 1H), 1.86 (ddd, J=3.2, 7.0, 10.9 Hz, 1H), 1.81-1.66 (m, 2H), 1.62 (ddd, J=3.9, 7.8, 15.8 Hz, 1H), 1.58-1.48 (m, 1H). ¹³C NMR (125 MHz, CDCl₃) δ 170.55, 144.63, 123.83, 69.70, 63.49, 57.20, 50.92, 47.30, 30.92, 27.43, 26.46, 24.84, 22.24. HRMS (ESI) calcd for [M+H]⁺: C₁₃H₂₀N₄O₂: 265.1659. Found: 265.1656.

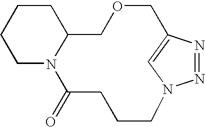

(5c) This compound was prepared using General Procedure I using 4-azido-1-(2-((prop-2-ynyloxy)methyl)piperidin-1-yl) but-1-one (220 mg, 0.832 mmol) Amberlyst-21 CuPF₆ (3.9 g, 0.832 mol, 0.21 mmol/g) in toluene (83 mL) to provide the title compound as a yellow oil (82 mg, 37% yield). IR (cm⁻¹) 2935, 2868, 2097, 1689, 1628, 1445, 1392, 1366, 1247, 1175, 1151, 1093, 1030. ¹H NMR (500 MHz, CDCl₃) δ 7.56 (s, 1H), 4.59 (d, J=13.3 Hz, 1H), 4.54-4.31 (m, 3H), 3.88-3.58 (m, 2H), 3.45-3.22 (m, 1H), 2.93-2.75 (m, 1H), 2.50-2.17 (m, 2H), 2.09-1.91 (m, 3H), 1.55 (t, J=15.1 Hz, 2H), 1.45-1.07 (m, 4H). ¹³C NMR (10:1 rotamer ratio, asterisk denotes minor rotamer peak, 125 MHz, CDCl₃) δ 171.60*, 170.93, 146.83, 145.27*, 124.56, 124.12*, 70.93, 69.48*, 64.90, 51.87, 51.08*, 50.06, 48.45*, 42.61, 36.70*, 28.36, 27.70, 26.59*, 25.95, 25.09, 23.91*, 20.17. HRMS (ESI) calcd for [M+H]⁺: C₁₃H₂₀N₄O₂: 265.1659. Found: 265.1662.

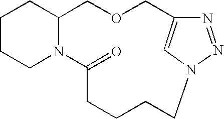

(5d) This compound was prepared using General Procedure I using 4-azido-1-(2-((prop-2-ynyloxy)methyl)piperidin-1-yl)pent-1-one (300 mg, 1.078 mmol) and Amberlyst-21 CuPF₆ (5.0 g, 1.078 mol, 0.21 mmol/g) in toluene (100 mL) to provide the title compound as a yellow oil (120 mg, 40% yield). IR (cm⁻¹) 3454, 3133, 2935, 2864, 2096, 1709, 1619, 1436, 1360, 1251, 1235, 1173, 1145, 1090, 1072, 1052, 1015, 968. ¹H NMR (500 MHz, DMSO at 130° C.) δ 7.83 (s, 1H), 4.60 (d, J=12.3 Hz, 1H), 4.51 (d, J=12.3 Hz, 1H), 4.38-4.30 (m, 1H), 4.29 (t, J=7.1 Hz, 2H), 4.17-4.07 (m, 1H), 3.76 (t, J=9.2, 1H), 3.58 (q, 1H), 2.82 (s, 1H), 2.73 (t, J=13.0 Hz, 1H), 2.40-2.26 (m, 2H), 1.88-1.79 (m, 2H), 1.74-1.67 (m, 1H), 1.64 (d, J=12.7 Hz, 1H), 1.59-1.46 (m, 6H), 1.38-1.26 (m, 1H). ¹³C NMR (125 MHz, DMSO at 130° C.) δ 171.7, 145.0, 123.6, 69.4, 64.8, 51.2, 49.9, 38.7, 32.3, 29.9, 26.6, 25.7, 22.5, 19.9. m.p. 235-238° C. HRMS (ESI) calcd for [M+H]⁺: C₁₃H₂₀N₄O₂: 265.1659. Found: 265.1660.

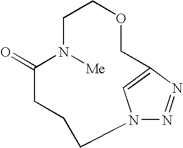

(5e) This compound was prepared using General Procedure I using 4-azido-N-methyl-N-(2-(prop-2-ynyloxy)ethyl)butanamide (135 mg, 0.602 mmol) Amberlyst-21 CuPF₆ (2.8 g, 0.602 mol, 0.21 mmol/g) in toluene (60 mL) to provide the title compound as a yellow oil (50 mg, 37% yield). IR (cm⁻¹) 3132, 2928, 2868, 1632, 1465, 1410, 1148, 1092, 1064, 732. ¹H NMR (300 MHz, CDCl₃) δ 7.52 (s, 1H), 4.68 (d, J=13.1 Hz, 1H), 4.56-4.39 (m, 2H), 4.32 (d, J=13.1 Hz, 1H), 3.81 (d, J=14.2, 1H), 3.70-3.61 (m, 2H), 2.92-2.75 (m, 1H), 2.69 (br s, 3H), 2.52-2.40 (m, 1H), 2.32-2.15 (m, 1H), 2.08-1.89 (m, 2H). ¹³C NMR (10:1 rotamer ratio, asterisk denotes minor rotamer peaks, 75 MHz, CDCl₃) δ 172.2, 171.7*, 146.0, 145.3*, 123.9, 123.5*, 67.7*, 66.5, 65.0*, 64.4, 51.4, 49.8, 49.7*, 38.5, 33.2*, 27.9, 27.5*, 24.5. HRMS (ESI) calcd for [M+H]⁺: C₁₀H₁₆N₄O₂: 225.1346. Found: 225.1353.

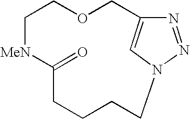

(5f) This compound was prepared using General Procedure I using 4-azido-N-methyl-N-(2-(prop-2-ynyloxy)ethyl)pentamide (200 mg, 0.834 mmol) Amberlyst-21 CuPF₆ (3.90 g, 0.834 mol, 0.21 mmol/g) in toluene (84 mL) to provide the title compound as a yellow oil (80 mg, 40% yield). IR (cm⁻¹) 3462, 3120, 3070, 2933, 2867, 1633, 1489, 1464, 1412, 1357, 1296, 1149, 1100. ¹H NMR (500 MHz, CDCl₃) δ 7.59 (s, 1H), 4.84 (d, J=13.3 Hz, 1H), 4.61 (t, J=12.6 Hz, 1H), 4.41-4.34 (m, 1H), 4.25 (d, J=13.3, 1H), 3.90 (d, J=14.2, 1H), 3.79 (d, J=11.3, 1H), 3.68 (dt, J=5.7, 11.3, 1H), 2.65 (s, 3H), 2.57 (ddd, J=7.1, 12.6, 22.5 Hz, 1H), 2.54-2.48 (m, 1H), 2.12-2.01 (m, 2H), 1.99-1.91 (m, 1H), 1.85-1.77 (m, 1H), 1.59-1.49 (m, 1H). ¹³C NMR (125 MHz, CDCl₃) δ 171.9, 144.8, 123.4, 67.2, 64.1, 50.9, 50.4, 38.8, 29.6, 26.5, 22.4. HRMS (ESI) calcd for [M+Na]⁺: C₁₁H₁₈N₄NaO₂: 261.1322. Found: 261.1332.

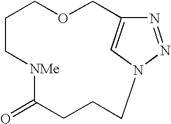

(5g) This compound was prepared using General Procedure I using 5-azido-N-methyl-N-(3-(prop-2-ynyloxy)propyl)pentanamide (135 mg, 0.567 mmol) Amberlyst-21 CuPF₆ (2.7 g, 0.567 mol, 0.21 mmol/g) in toluene (57 mL) to provide the title compound as a yellow oil (65 mg, 48% yield). IR (cm⁻¹) 3126, 2924, 2862, 2241, 2097, 1651, 1450, 1424, 1407, 1253, 1224, 1183, 1137, 1121, 1104, 1058, 1043, 1026, 964, 908, 870, 846, 770. ¹H NMR (1.2:1 rotamer ratio, asterisk denotes minor rotamer peaks, 500 MHz, CDCl₃) δ 7.61 (s, 1H), 7.60*(s, 1H), 4.69 (t, J=13.4 Hz, 1H), 4.52 (dt, J=4.0, 13.0 Hz, 1H), 4.37 (d, J=11.2 Hz, 1H), 4.35-4.30 (m, 1H), 4.05 (d, J=11.0 Hz, 1H), 3.75-3.68 (m, 1H), 3.63-3.58 (m, 1H), 3.51-3.44*(m, 1H), 3.37-3.28*(m, 1H), 2.87 (s, 3H), 2.65*(s, 3H), 2.63-2.61*(m, 1H), 2.56 (d, J=13.7 Hz, 1H), 2.39-2.31 (m, 1H), 2.29-2.20*(m, 1H), 2.14-2.06*(m, 3H), 2.04-1.93 (s, 3H), 1.68-1.58 (m, 1H), 1.48 (d, J=13.4 Hz, 1H). ¹³C NMR (1.1:1 rotamer ratio, asterisk denotes minor rotamer peaks, 125 MHz, CDCl₃) δ 172.9*, 171.6, 134.6*, 134.1, 133.6*, 133.5, 72.0*, 67.6, 60.2*, 60.1, 47.2*, 46.3, 45.9, 45.6*, 34.6*, 32.4, 27.7, 27.3*, 26.6*, 26.3, 25.5, 24.8*. HRMS (ESI) calcd for [M+H]⁺: C₁₁H₁₈N₄O₂: 239.1503. Found: 239.1507.

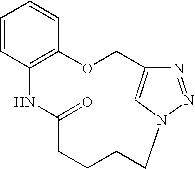

(5h) This compound was prepared using General Procedure I using 4-azido-N-methyl-N-(2-(prop-2-ynyloxy)phenyl)butanamide (200 mg, 0.734 mmol) Amberlyst-21 CuPF₆ (3.50 g, 0.734 mol, 0.21 mmol/g) in toluene (73 mL) to provide the title compound as a yellow oil (61 mg, 31% yield). IR (cm⁻¹) 3344, 3144, 2939, 1671, 1596, 1519, 1447, 1300, 1250, 1181, 1107, 957, 836, 787, 772, 741, 667. ¹H NMR (500 MHz, CDCl₃) δ 8.01-7.97 (m, 1H), 7.40 (s, 1H), 7.19-7.16 (m, 1H), 7.14-7.09 (m, 2H), 6.82 (s, 1H), 5.16 (s, 2H), 4.49 (t, J=5.5 Hz, 2H), 2.07-2.01 (m, 2H), 1.95-1.88 (m, 2H), 1.87-1.81 (m, 2H). ¹³C NMR (126 MHz, CDCl₃) δ 170.6, 149.8, 145.5, 131.5, 125.5, 125.2, 123.0, 122.5, 69.7, 51.5, 33.8, 27.1, 22.9. HRMS (ESI) calcd for [M+Na]⁺: C₁₄H₁₆N₄NaO₂: 295.2922. Found: 295.1165.

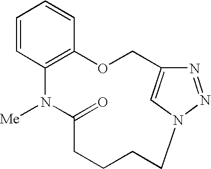

(5i) This compound was prepared using General Procedure I using 4-azido-N-(2-(prop-2-ynyloxy)phenyl)butanamide (200 mg, 0.698 mmol) Amberlyst-21 CuPF₆ (3.3 g, 0.698 mol, 0.21 mmol/g) in toluene (70 mL) to provide the title compound as a white solid (129 mg, 65% yield). IR (cm⁻¹) 3122, 3065, 2935, 1647, 1496, 1456, 1423, 1382, 1352, 1257, 1234, 1218, 1200, 1136, 1093, 1042, 972, 911, 773, 729. ¹H NMR (500 MHz, CDCl₃) δ 7.66 (s, 1H), 7.42 (d, J=1.5, 8.3 Hz, 1H), 7.34 (ddd, J=1.5, 7.0, 8.5 Hz, 1H), 6.94 (dt, J=1.5, 7.6 Hz, 1H), 6.84 (dd, J=1.6, 7.8 Hz, 1H), 5.41 (d, J=13.0, 1H), 5.27 (d, J=13.1, 1H), 4.50 (ddd, J=3.0, 11.0, 11.5 Hz, 1H), 4.28 (dt, J=4.0, 14.1 Hz, 1H), 3.14 (s, 3H), 2.24-2.33 (m, 1H), 2.01-2.09 (m, 1H), 1.95-1.82 (m, 3H), 1.44-1.54 (m, 1H), −0.15 (ddd, J=5.0, 6.5, 18.0 Hz, 1H). ¹³C NMR (125 MHz, CDCl₃) δ 172.0, 152.6, 145.0, 135.4, 129.6, 129.0, 123.9, 123.5, 122.2, 65.4, 51.8, 36.3, 31.2, 26.2, 22.8. m.p.=214-217° C. HRMS (ESI) calcd for [M+H]⁺: C₁₅H₁₈N₄O₂: 287.1503. Found: 287.1503.

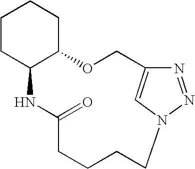

(5j) This compound was prepared using General Procedure I using 5-azido-N-((trans)-2-hydroxycyclohexyl)pentanamide (160 mg, 0.575 mmol) Amberlyst-21 CuPF₆ (2.5 g, 0.575 mol, 0.21 mmol/g) in toluene (55 mL) to provide the title compound as a white solid (28 mg, 17% yield). IR (cm⁻¹): 3298 (br), 2929, 2858, 1655, 1545, 1448, 1078, 729. ¹H NMR (500 MHz, CDCl₃) δ 7.88 (br s, 1H), 4.85 (s, 1H), 4.65 (s, 1H), 4.53 (s, 2H), 4.42 (s, 1H), 3.43 (s, 1H), 3.23 (s, 1H), 2.15 (m, 3H), 1.96 (m, 2H), 1.83 (d, J=10.5 Hz, 1H), 1.72 (s, 1H), 1.59 (m, 3H), 1.34 (m, 1H), 1.18 (m, 4H). ¹³C NMR_ (4:1 rotamer ratio, asterisk denotes minor rotamer peaks, 125 MHz, CDCl₃) δ 170.82, 83.70, 83.64*, 64.74, 53.28, 53.19*, 51.34, 33.24, 32.73, 32.70*, 32.24, 32.19*, 26.98, 24.46, 24.40, 21.93. m.p. 221-223° C. HRMS (ESI) calcd for [M+H]⁺: C₁₄H₂₂N₄O₂: 279.1816. Found: 279.1814.

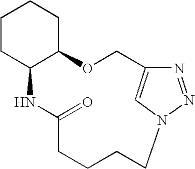

(5k) This compound was prepared using General Procedure I using 5-azido-N-((cis)-2-hydroxycyclohexyl)pentanamide (200 mg, 0.719 mmol) Amberlyst-21 CuPF₆ (3.6 g, 0.719 mol, 0.21 mmol/g) in toluene (65 mL) to provide the title compound as a white solid (125 mg, 63% yield). IR (cm⁻¹): 3323 (br), 2929, 2858, 1643, 1533, 1069, 1047, 731. ¹H NMR (500 MHz, CDCl₃) δ 7.54 (s, 1H), 4.90 (d, J=13.4, 1H), 4.72 (d, J=8.5 Hz, 1H), 4.61 (ddd, J=2.0, 12.0, 14.0 Hz, 1H), 4.40 (ddd, J=2.0, 5.0, 14.0 Hz, 1H), 4.23 (d, J=13.4, 1H), 3.70 (m, 1H), 3.51 (m, 1H), 2.22-1.99 (m, 4H), 1.89 (d, J=13.4 Hz, 1H), 1.73-1.63 (m, 1H), 1.55 (m, 2H), 1.47-1.31 (m, 5H), 1.30-1.20 (m, 1H). ¹³C NMR (125 MHz, CDCl₃) δ 170.4, 146.0, 122.4, 74.8, 62.2, 50.9, 49.2, 31.6, 28.9, 26.9, 26.8, 24.6, 22.6, 19.2. m.p. 208-209° C. HRMS (ESI) calcd for [M+H]⁺: C₁₄H₂₂N₄O₂: 279.1816. Found: 279.1817.

### Example 6

### Synthesis of Amberlyst-21 CuPF₆

To a suspension of Amberlyst-21 (140.1 g, 938 mmol) in CH₃CN (800 mL) was added to a solution of Tetrakis(acetonitrile) copper hexafluorophospate (21.19 g, 56.9 mmol) in CH₃CN (200 mL). The mixture was gently shaken using vortex mixer for 1 h at RT. The solvent was filtered and the beads were washed with CH₃CN (3×800 mL), dichloromethane (3×800 mL) and dried under vacuum at 40° C. for 16 h to yield 147 g of resin (PS—CuPF₆, loading=0.21 mmol/g).

### Incorporation by Reference

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of forming a triazole according to Scheme 1: 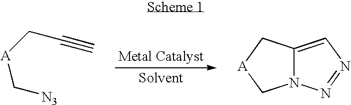
wherein, independently for each occurrence,
A is -(a)ₘ-;
metal catalyst consists essentially of at least one ligand and Ru;
a represents —O—, —NR—, —C(═O)—, —CR₂—, —S—, —RP(═O)—, —S(═O)—, —SO₂—, or phenyl;
m is 6, 7, 8, 9, 10, 11, or 12; and
R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

2. The method of claim 1, wherein -(a)ₘ- comprises an amide.

3. The method of claim 2, wherein m is 7, 8, or 9.

4. The method of claim 1, wherein A is —O—(CR₂)₂—NR—C(═O)—(CR₂)₂—, —O—(CR₂)₂—NR—C(═O)—(CR₂)₃—, or —O—(CR₂)₃—NR—C(═O)—(CR₂)₂—.

5. The method of claim 1, wherein A is selected from the group consisting of

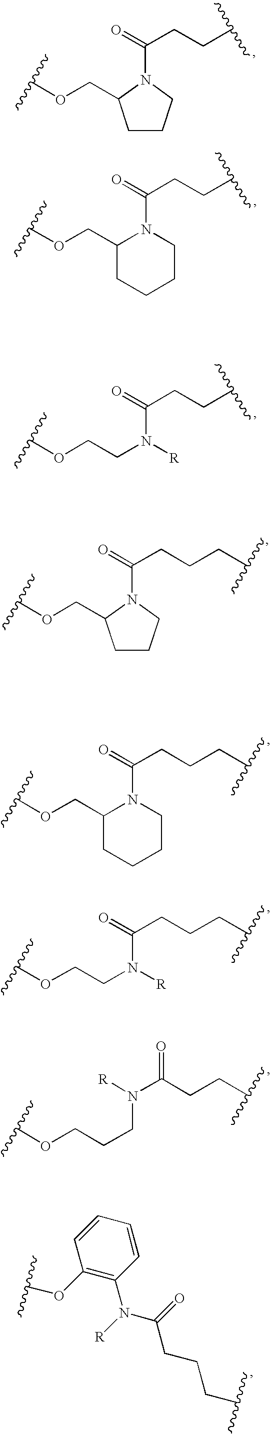

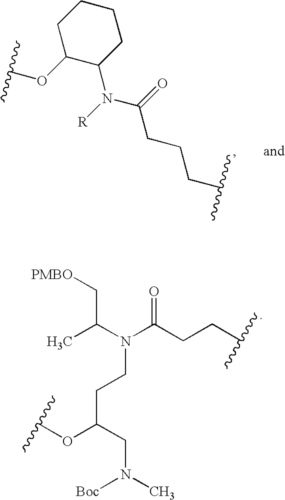

6. The method of claim 1, wherein the metal catalyst is [Cp*RuCl]₄, Cp*RuCl(COD), or Cp*RuCl(PPh₃).

7. A method of forming a triazole according to Scheme 2: 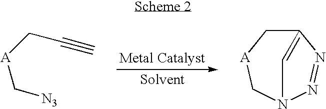
wherein, independently for each occurrence,
A is -(a)ₘ-;
metal catalyst consists essentially of at least one ligand and Cu;
a represents —O—, —NR—, —C(═O)—, —CR₂—, —S—, —RP(═O)—, —S(═O)—, —SO₂—, or phenyl;
m is 6, 7, 8, 9, 10, 11, or 12; and
R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂—C(═O)OH, and —C(═NH)NH₂.

8. The method of claim 7, wherein -(a)ₘ- comprises an amide.

9. The method of claim 8, wherein m is 7, 8, or 9.

10. The method of claim 7, wherein A is —O—(CR₂)₂—NR—C(═O)—(CR₂)₂—, —O—(CR₂)₂—NR—C(═O)—(CR₂)₃—, or —O—(CR₂)₃—NR—C(═O)—(CR₂)₂—.

11. The method of claim 7, wherein A is selected from the group consisting of

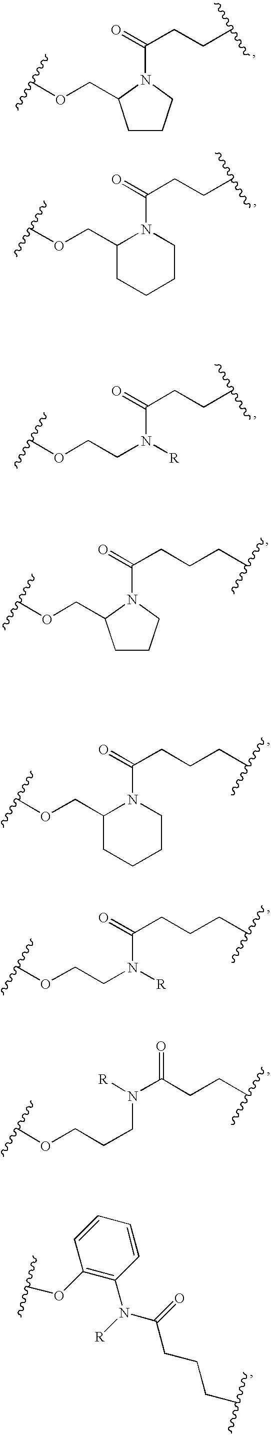

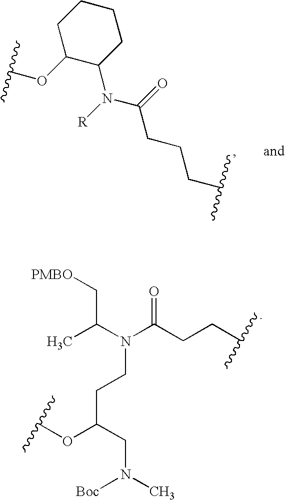

12. The method of claim 7, wherein the metal catalyst is Cu (CH₃CN)₂PF₆, (CN)₄CuPF₆, CuI PS—N(CH₃)₂CuI, or PS—N(CH₃)₂CuPF₆.

13. A compound of formula I 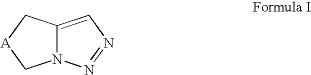
or a pharmaceutically acceptable salt thereof,
wherein, independently for each occurrence,
A is -(a)ₘ-;
a represents —O—, —NR—, —C(═O)—, —CR₂—, —S—, —RP(═O)—, —S(═O)—, —SO₂—, or phenyl;
m is 6, 7, 8, 9, 10, 11, or 12; and
R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

14. The compound of claim 13, wherein -(a)ₘ- comprises an amide.

15. The compound of claim 14, wherein m is 7, 8, or 9.

16. The compound of claim 13, wherein A is —O—(CR₂)₂—NR—C(═O)—(CR₂)₂—, —O—(CR₂)₂—NR—C(═O)—(CR₂)₃—, or —O—(CR₂)₃—NR—C(═O)—(CR₂)₂—.

17. The compound of claim 13, wherein A is selected from the group consisting of

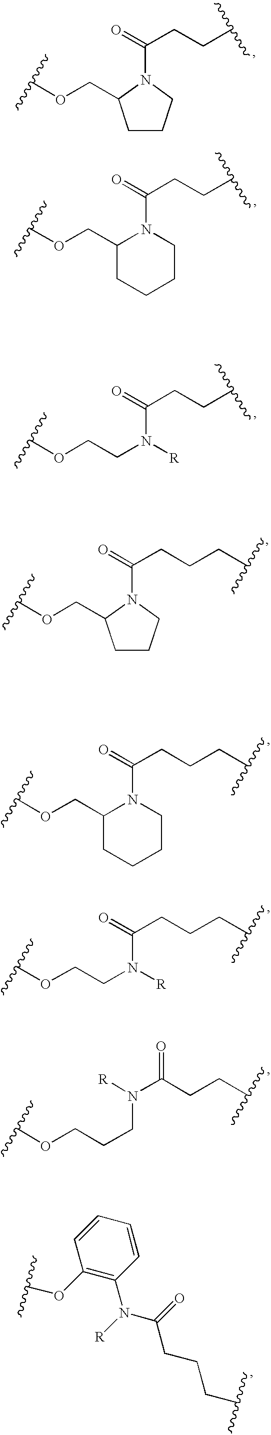

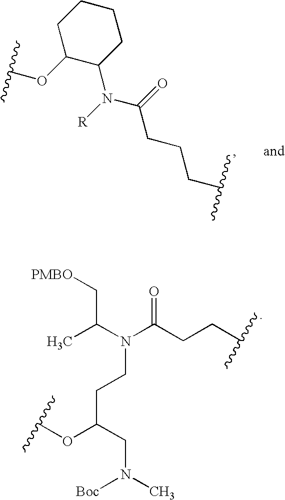

18. The compound of claim 13 selected from the group consisting of

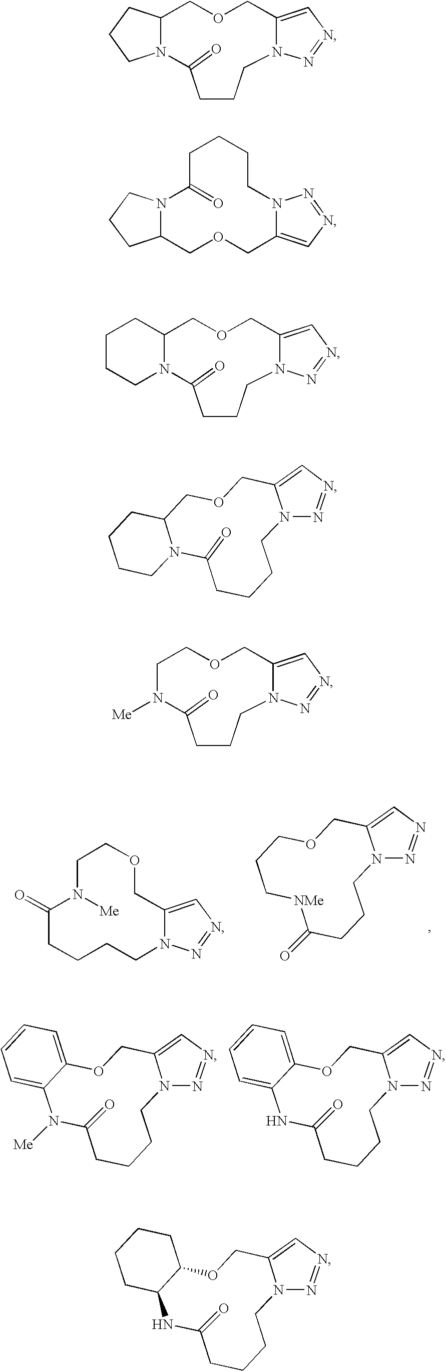

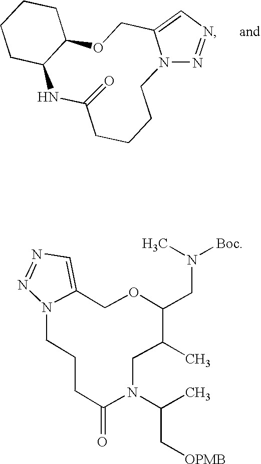

19. A compound of formula II 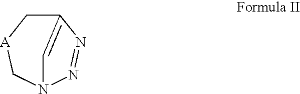
or a pharmaceutically acceptable salt thereof,
wherein, independently for each occurrence,
A is -(a)ₘ-;
a represents —O—, —NR—, —C(═O)—, —CR₂—, —S—, —RP(═O)—, —S(═O)—, —SO₂—, or phenyl;
m is 6, 7, 8, 9, 10, 11, or 12; and
R is hydrogen, alkyl, hydroxy, halo, cyano, amino, aminoalkyl, alkenyl, cycloalkyl, aryl, aralkyl, acetyl, formyl, sulfonyl, alkoxy, alkenyloxy, aryloxy, arylalkyloxy, nitro, SH, amido, or sulfonamido; or two instances of R, taken together with the atoms to which they are attached, form a 5-, 6-, or 7-membered cycloalkyl, heterocycloalkyl, aryl, or heteroaryl ring; or two adjacent instances of R, taken together, form a double bond between the atoms to which they are attached;
wherein any one of the aforementioned alkoxy, alkenyloxy, aryloxy, aralkyloxy, alkyl, alkenyl, cycloalkyl, aryl, aminoalkyl, and aralkyl groups may be optionally substituted with one or more groups selected from the group consisting of hydroxy, alkoxy, alkyl, acyl, alkyloxycarbonyl, alkenyloxy, aryloxy, aralkyloxy, halo, formyl, acetyl, cyano, nitro, SH, amino, amido, sulfonyl, sulfonamido, —S-alkyl, —C(═O)NH₂, —C(═O)OH, and —C(═NH)NH₂.

20. The compound of claim 19, wherein -(a)ₘ- comprises an amide.

21. The compound of claim 20, wherein m is 7, 8, or 9.

22. The compound of claim 19, wherein A is —O—(CR₂)₂—NR—C(═O)—(CR₂)₂—, —O—(CR₂)₂—NR—C(═O)—(CR₂)₃—, or —O—(CR₂)₃—NR—C(═O)—(CR₂)₂—.

23. The compound of claim 19, wherein A is selected from the group consisting of

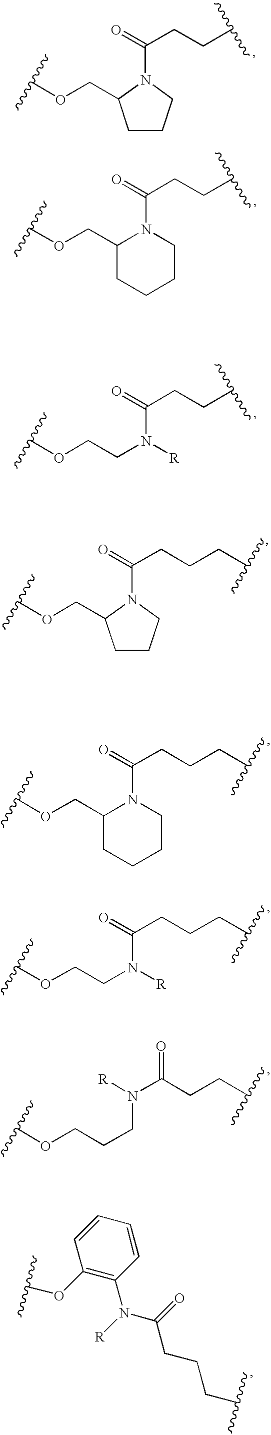

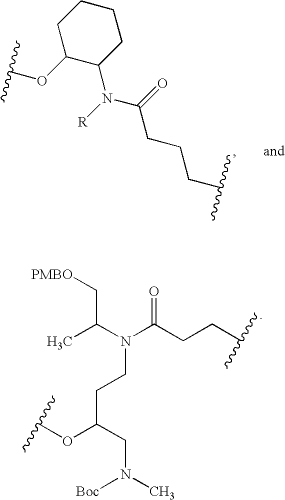

24. The compound of claim 19 selected from the group consisting of [chemical structure]
